(19) United States
(12) Patent Application Publication
(10) Pub. No.: US 2023/0053353 A1
(43) Pub. Date: Feb. 23, 2023

(54) TARGETING TRANSFER RNA FOR THE SUPPRESSION OF NONSENSE MUTATIONS IN MESSENGER RNA

(71) Applicant: The Regents of the University of California, Oakland, CA (US)

(72) Inventors: Prashant Mali, La Jolla, CA (US); Dhruva Katrekar, La Jolla, CA (US); Thomas Morton, La Jolla, CA (US)

(21) Appl. No.: 17/791,498

(22) PCT Filed: Jan. 8, 2021
(86) PCT No.: PCT/US2021/012806

§ 371 Date: Jul. 7, 2022

Related U.S. Application Data
(60) Provisional application No. 62/959,087, filed on Jan. 9, 2020.

(51) Int. Cl. C12N 15/11; C12N 15/63; C12N 15/10

(52) U.S. Cl. CPC C12N 15/11; C12N 15/63; C12N 15/1024; C12N 2310/16; C12N 2310/122

(57) ABSTRACT

The disclosure provides for a targeting transfer RNA (ttRNA) that that suppresses nonsense mutations in messenger RNA, that comprises an anticodon sequence that binds to a stop codon and a variable loop sequence that comprises an RNA aptamer that has strong binding affinity to an RNA binding protein; and methods of use thereof.

### CROSS REFERENCE TO RELATED APPLICATIONS

This application claims priority under 35 U.S.C. § 119 from Provisional Application Ser. No. 62/959,087, filed Jan. 9, 2020, the disclosures of which are incorporated herein by reference.

### STATEMENT OF GOVERNMENT SUPPORT

This invention was made with Government support under Grant Nos. RO1HG009285, RO1CA222826, and RO1GM123313, awarded by the National Institutes of Health. The Government has certain rights in the invention.

### TECHNICAL FIELD

The disclosure provides for tRNA that suppresses nonsense mutations in messenger RNA, and uses thereof.

### INCORPORATION BY REFERENCE OF SEQUENCE LISTING

Accompanying this filing is a Sequence Listing entitled, “Sequence-Listing_ST25” created on Jan. 8, 2021 and having 15,197 bytes of data, machine formatted on IBM-PC, MS-Windows operating system. The sequence listing is hereby incorporated by reference in its entirety for all purposes.

### BACKGROUND

Transfer RNA (tRNA) are an integral component of the translation process. tRNA's primary role is to shuttle single amino acids to the ribosomal/mRNA complex. Prior to arrival, the tRNA will interact with a synthetase which charges the tRNA with a particular amino acid. Then it will bind to eukaryotic elongation factor 1a (eEF-1a) for assistance in delivery to the ribosome. Upon arrival, it will interact with a particular nucleotide triplet, known as codon, and deposit its amino acid. The ribosome attaches this amino acid to the growing peptide chain, ultimately synthesizing a functional protein specific to the particular mRNA through a process called translation. The proteins produced during the translational process are largely dictated by the particular sequence of codons found on the mRNA. However, mutations of the DNA can also produce mutated mRNA during transcription. These mutations can take various forms, including nonsense mutations. Nonsense mutations are single nucleotide mutations that convert a codon into one of three stop codons; UAG, UAA, or UGA. These premature termination codons (PTCs) result in a truncated version of a protein, which is often no longer functional. Nonsense mutations can cause a genetic disease by preventing complete translation of a specific protein. Examples of such diseases include Duchenne muscular dystrophy, cystic fibrosis, Beta thalassaemia (β-globin), Hurler syndrome, and Dravet Syndrome.

### SUMMARY

Therapeutic opportunities are possible with the suppressor tRNA approach, however, efficiency, specificity, and delivery of suppressor tRNA is subpar. The disclosure provides for targeted transfer tRNAs (ttRNAs), which provides significant improvements in efficiency, specificity, and delivery of suppressor tRNA. ttRNAs were created through the use of mutagenesis, and by targeting two areas of the tRNA: the tRNA's anticodon loop was modified to interact with UAG stop codons; and the variable loop of the tRNA was replaced with known RNA aptamers that have strong binding affinity to specific RNA binding proteins. These RNA aptamers are single stranded sequences that form RNA hairpin loops, which interact with binding pockets of RNA binding proteins known as RNA Recognition Motifs (RRMs). As shown in FIG. 3, the ttRNAs can be combined with specifically engineered guide RNAs (gRNAs) to allow for localization of the ttRNA to specific mRNAs of interest, thereby minimizing off target suppression of native stop codons in vicinity of the 3′ UTR, and increasing the efficiency of PTC suppression in general.

The present disclosure provides a targeting transfer RNA (ttRNA) comprising: a polynucleotide having a general structure from 5′ to 3′ of: 5′-(D-Loop domain)-(anticodon loop domain)-(variable loop domain)-(T loop domain)-3′, wherein the anticodon loop domain comprises a sequence selected from X₁UCTAX₂X₃; X₁UUUAX₂X₃; or X₁UUCAX₂X₃, wherein X₁ is a pyrimidine, X₂ is a purine and X₃ is any of U, A or C, that binds to a stop codon sequence of an mRNA, wherein the variable loop domain is about 12-30 nucleotides in length and which comprises a sequence that is selectively bound by a protein or an agent tag, and wherein the ttRNA suppresses nonsense mutations in messenger RNA. The ttRNA can comprise a nucleic acid sequence that binds with an RNA binding protein. The ttRNA can be engineered from a tRNA for an amino acid selected from the group consisting of alanine, asparagine, aspartic acid, arginine, cysteine, glutamine, glycine, glutamic acid, histidine, isoleucine, lysine, leucine, phenylalanine, proline, methionine, serine, tryptophan, threonine, tyrosine, and valine. The polynucleotide can be derived from a tRNA for serine or arginine. The tRNA can be found in humans. The ttRNA can have a cloverleaf-like structure. The D-Loop domain and T-Loop domain of the polynucleotide can comprise a naturally occurring sequence from a human tRNA. The anticodon loop domain can bind to a stop codon having a DNA sequence of ‘TAG’ or an RNA sequence of ‘UAG.’ The anticodon loop domain can bind to a stop codon having a DNA sequence of ‘TAA’ or an RNA sequence of ‘UAA.’ The anticodon loop domain can bind to a stop codon having a DNA sequence of ‘TGA’ or an RNA sequence of ‘UGA.’ The variable loop domain can comprise a sequence that is selectively bound by an RNA binding protein. The variable loop domain can comprise a sequence for TBP, BoxBv1, BoxBv2, MS2v2, U1Av3, Gas5v4, TARv1, SLBPv1, or SLBPv2. The variable loop domain can comprise a sequence for BoxBv1. The variable loop domain can comprise a sequence with at least 70%, 80%, 90% or 99% sequence identity to a sequence selected from the group consisting of GGCCCTGAAAAAGGGCC (SEQ ID NO:21), GGGACATGAGGATCACCCATGTCCC (SEQ ID NO:22), AGCTTATCCATTGCACTCCGGATAAGCT (SEQ ID NO:23), GGCCCAGTGGTCTTTGTAGACTGCCTGATGGCC (SEQ ID NO:24), GGCCAGATCTGAGCCTGGGAGCTCTCTGGCC (SEQ ID NO:25), CCAAAGGCTCTTCTCAGAGCCACCCA (SEQ ID NO:26), and GGCTCTTCTCAGAGCC (SEQ ID NO:27). The polynucleotide can comprise a sequence with at least 70%, 80%, 90% or 99% sequence identity to a sequence selected from (1) to (8): (1) GTAGTCGTGGCCGAGTGGTTAAGGCGATGGACTCTAAATCCATTGGCCC TGAAAAAGGGCCGCGCAGGTTCGAATCCTGCCGACTACG; (2) GTAGTCGTGGCCGAGTGGTTAAGGC GATGGACTCTAAATCCATTCGGCCCTGAAAAAGGGCCGCGCAGGTTCGAATCCTGCCGACTACG; (3) GTAGTCGTGGCCGAGTGGTTAAGGCGATGGACTCTAAATCCATTGGGGACATGAGGATCACCCATGTC CCGCGCAGGTTCGAATCCTGCCGACTACG; (4) GTAGTCGTGGCCGAGTGGTTAAGGCGATGGACTCT AAATCCATTAGCTTATCCATTGCACTCCGGATAAGCTGCGCAGGTTCGAATCCTGCCGACTACG; (5) GTAGTCGTGGCCGAGTGGTTAAGGCGATGGACTCTAAATCCATTGGCCCAGTGGTCTTTGTAGACTGC CTGATGGCCGCGCAGGTTCGAATCCTGCCGACTACG; (6) GTAGTCGTGGCCGAGTGGTTAAGGCGAT GGACTCTAAATCCATTGGCCAGATCTGAGCCTGGGAGCTCTCTGGCCGCGCAGGTTCGAATCCTGCCG ACTACG; (7) GTAGTCGTGGCCGAGTGGTTAAGGCGATGGACTCTAAATCCATTCCAAAGGCTCTTCT CAGAGCCACCCAGCGCAGGTTCGAATCCTGCCGACTACG; and (8) GTAGTCGTGGCCGAGTGGTT AAGGCGATGGACTCTAAATCCATTGGCTCTTCTCAGAGCCGCGCAGGTTCGAATCCTGCCGACTACG. The polynucleotide can comprise the sequence of: gtagtcgtggccgagtggt taaggcgatggactctaaatccattggccctgaaaaagggccgcgcaggttcgaatcctgccgactac g.

The present disclosure also provides a vector that can encode a ttRNA. The vector can be a viral vector. The viral vector can be a retroviral vector or an adeno-associated viral vector. The present disclosure also provides a composition, for which in some instances can be used for at least partially suppressing nonsense mutations in messenger RNA comprising: the ttRNA, or the vector; and a guide RNA (gRNA) that specifically binds to a nucleotide sequence within 20-100 bps of a nonsense mutation, wherein the gRNA further comprises one or more RNA binding protein sequences that have strong binding affinity for one or more RNA binding protein(s); and one or more complexes comprising two RNA binding proteins that are fused together, wherein a first RNA binding protein of a complex has strong binding affinity for one or more RNA binding protein sequences of the gRNA, and wherein a second RNA binding protein of a complex has strong binding affinity for a sequence in the variable loop domain of the ttRNA. The one or more complexes can comprise two RNA binding proteins selected from the group consisting of LN, MCP, U1A, GRD-BD, TBP6.7, SLBP and variants thereof. The one or more complexes can comprise TBP and SLBPx3 fused together. The one or more complexes comprise MCP and LNx4 fused together.

The present disclosure also provides a method, for which in some instances can be used for at least partially restoring translation in a cell of a nucleotide sequence that has a nonsense mutation comprising: contacting the cell with a ttRNA, a vector, or a composition. The cell can be contacted in vivo, in vitro or ex vivo. The cell can be from a subject that has a disorder associated with a nonsense mutation. The subject can have a disorder selected from the group consisting of Duchenne muscular dystrophy, cystic fibrosis, Beta thalassaemia (β-globin), Hurler syndrome, and Dravet Syndrome. The subject can have Duchenne muscular dystrophy or cystic fibrosis.

The present disclosure also provides an engineered targeting transfer RNA (ttRNA) comprising a modified variable loop, wherein the modified variable loop comprises a hairpin loop that binds to an RNA binding protein to a greater extent than a tRNA comprising an unmodified variable loop. The tRNA comprising the unmodified variable loop can be naturally present in a human cell. The hairpin loop can comprise at least a portion of an aptamer. The hairpin loop can comprise at least a portion of an MS2 domain. The hairpin loop can comprise at least a portion of a BoxB domain. The hairpin loop can comprise at least a portion of a U1hpII domain. The hairpin loop comprises at least a portion of a Gas5 domain. The RNA binding protein is selected from the group consisting of at least a portion of: Lambda N, MCP, U1A, GR-DBD, and any combination thereof. The engineered ttRNA can be acylated with an amino acid. The amino acid can be a canonical amino acid. The amino acid cannot be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or non-canonical amino acid. The engineered ttRNA can comprise an anticodon loop that recognizes a stop codon implicated in a disease or condition. The stop codon can be a premature stop codon. The stop codon can be UAA. The stop codon can be UGA. The stop codon can be UAG. The engineered ttRNA when contacted with the target RNA at least partially disrupts association of a release factor protein with the target RNA. The engineered ttRNA comprises an anticodon loop that recognizes a sense codon, wherein the sense codon comprises the mutation in the target RNA that is implicated in the disease or condition. The sense codon can be GCU, GCC, GCA, GCG, CGU, CGC, CGA, CGG, AGA, AGG, AAU, AAC, GAU, GAC, UGU, UGC, GAA, GAG, CAA, CAG, GGU, GGC, GGA, GGG, CAU, CAC, AUU, AUC, AUA, UUA, UUG, CUU, CUC, CUA, CUG, AAA, AAG, AUG, UUU, UUC, CCU, CCC, CCA, CCG, UCU, UCC, UCA, UCG, AGU, AGC, ACU, ACC, ACA, ACG, UGG, UAU, UAC, GUU, GUC, GUA, or GUG. The engineered ttRNA when contacted with the target RNA at least can partially disrupt association of a tRNA naturally present in a human with the target RNA, wherein the tRNA naturally present in the human cell comprises an anticodon loop that is complementary to the sense codon. The at least one nucleotide of the engineered ttRNA can be a chemically modified nucleotide. The engineered ttRNA can comprise a sugar modification. The nucleotide of the engineered ttRNA comprises a methyl group, a fluoro group, a methoxyethyl group, an ethyl group, a phosphate group, an amide group, an ester group, or any combination thereof. The engineered ttRNA can be genetically encodable. The engineered ttRNA can comprise a reducing 3′ hydroxyl group. The modified variable loop can comprise a hairpin loop that binds to an RNA binding protein to a greater extent than a tRNA comprising an unmodified variable loop is determined by an in vitro assay. The disease or condition comprises Rett Syndrome, Duchenne Muscular Dystrophy, Stargardt's Syndrome, Alzheimer's diseases, Parkinson's disease, or any combination thereof

A system comprising: the engineered ttRNA, a first RNA binding protein, and a guide RNA comprising an antisense domain, wherein the antisense domain is complementary to at least a portion of a target RNA comprising a mutation, and wherein the mutation of the target RNA is implicated in a disease or condition. The engineered ttRNA further comprises a hairpin loop that binds to a second RNA binding protein. The first RNA binding protein or the second RNA binding protein is selected from the group consisting of at least a portion of: Lambda N, MCP, U1A, GR-DBD, and any combination thereof. The guide RNA can comprise a hairpin capable of binding the first RNA binding protein. The hairpin can comprise an BoxB hairpin. The engineered ttRNA can be capable of binding the first RNA binding protein. The hairpin can comprise a MS2 hairpin. The first RNA binding protein can comprise a first binding domain that binds a second binding domain of the second RNA binding protein. The first RNA binding protein can bind the second RNA binding protein with a K_{D} of from about 1 nM to about 100 μM. The guide RNA can form a secondary structure comprising: a stem loop, a cruciform, a toe hold, a mismatch, or any combination thereof. The RNA binding protein can be delivered exogenously. The RNA binding protein can be endogenously expressed.

The present disclosure provides a vector comprising a polynucleotide sequence encoding for the engineered ttRNA or the engineered ttRNA and guide RNA of the system. The vector can comprise a liposome, a viral vector, a nanoparticle, or any combination thereof. The vector can be the viral vector, and wherein the viral vector is an AAV vector.

The present disclosure also provides an isolated cell that comprises the engineered ttRNA of the present disclosure, the system of the present disclosure, or the vector of the present disclosure.

The present disclosure also provides a pharmaceutical composition in unit dose form comprising: the engineered ttRNA of the present disclosure, the engineered ttRNA and guide RNA of the system of the present disclosure, or the vector of the present disclosure, and a pharmaceutically acceptable: excipient, diluent, or carrier.

The present disclosure also provides a method of at least partially ameliorating or preventing a disease or condition in a subject in need thereof comprising: administering to the subject the engineered ttRNA of the present disclosure, the engineered ttRNA and guide RNA of the system of the present disclosure, the vector of the present disclosure, or the pharmaceutical composition of the present disclosure, and wherein the administering is sufficient at least partially ameliorate the disease or condition in the subject. The administering can be by intravenous injection, intramuscular injection, an intrathecal injection, an intraorbital injection, a subcutaneous injection, or any combination thereof. The disease or condition can be selected from the group consisting of: a neurodegenerative disorder, a muscular disorder, a metabolic disorder, an ocular disorder, a cancer, and any combination thereof. The disease or condition can comprise Rett Syndrome. The subject can be a mammal. The mammal can be a human. The mammal can be a non-human mammal. The subject can be diagnosed with the disease or condition by an in vitro diagnostic.

The present disclosure provides a kit comprising: the engineered ttRNA of the present disclosure in a container, the engineered ttRNA and guide RNA of the system of the present disclosure in a container, or the vector of the present disclosure in a container.

The present disclosure also provides a method of making a kit, comprising placing at least in part, into a container: the engineered ttRNA of the present disclosure, the engineered ttRNA and guide RNA of the system of the present disclosure, or the vector of the present disclosure. The present disclosure provides a method of making a pharmaceutical composition, comprising contacting a pharmaceutically acceptable: excipient, carrier, or diluent with at least one of the engineered ttRNA of the present disclosure, the engineered ttRNA and guide RNA of the system of the present disclosure, or the vector of the present disclosure.

### DESCRIPTION OF DRAWINGS

FIG. 1A-B provides schematics of how a PTC suppressor tRNA system of the disclosure works in rescuing translation. (A) The mRNA (SEQ ID NO:1) has a PTC boxed in red. When the ribosome reaches the PTC, it disengages from the mRNA, resulting in a truncated protein compared to the expected protein (SEQ ID NO:2). (B) The same PTC exists in this mRNA (SEQ ID NO:1), except the engineered suppressor ttRNA interacts with the PTC, allowing the ribosome to continue translation to provide the full protein (SEQ ID NO:2).

FIG. 2 provides a plot of the highest normalized (to the NC) Nluc signal recovery for each suppressor tRNA family and PTC type.

FIG. 3A-B provides a schematic of the ttRNA system which includes the ttRNA, gRNA, RNA hairpin, and RNA binding proteins in order to target a specific mRNA of interest.

FIG. 4 shows visualization of modifications to the anticodon loop of a selected tRNA sequence. (Above panel) Core serine sequences (SEQ ID NO:3) used for the native and suppressor versions of the tRNA. (Below panel) Secondary structure visualization of the tRNA and modification to anti-codon loop (SEQ ID NO:4).

FIG. 5 presents a Table of most current and tested ttRNASer-TAG constructs with associated sequences (SEQ ID NOs: 5-12).

FIG. 6 presents secondary structure visualizations of the ttRNASer-TAG constructs and RNA hairpin modifications to the variable loop (SEQ ID NOs: 4-12).

FIG. 7A-G provides transfection experiments in HEK239 cells with the various plasmid constructs. (A) Positive Control consisting of GFP and mCherry reporters. (B) Negative Control consisting of GFP (TAG PTC) and mCherry. (C) GFP (TAG PTC) and ttRNASer-TAG. (D) GFP (TAG PTC) and ttRNASer-TAG+BoxBv1. (E) GFP (TAG PTC) and ttRNASer-TAG+MS2v1. (F) GFP (TAG PTC) and ttRNASer-TAG+U1Av1. (G) Provides a schematic and assay structure used in the disclosure.

FIG. 8 provides a plot of normalized (to PC) cLuc signal recovery for each suppressor ttRNA+RNA hairpin construct.

FIG. 9 provides a plot of normalized (to PC) cLuc signal recovery for each suppressor ttRNA+RNA hairpin construct.

FIG. 10 provides a plot of normalized cLuc and gLuc signal recovery for each suppressor ttRNA and recruitment system components. PCs represent cLuc and gLuc reporters' absent PTCs. NCs are the reporters with a PTC at S56X for cLuc and S78X for gLuc. Condition 1 contains all of the recruitment components. Condition 2 contains just the suppressor ttRNA.

FIG. 11 provides the sequence (SEQ ID NO:13) and structure for cLuc gRNA with MS2 hairpin.

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the mutation” includes reference to one or more mutations and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

As used herein, the term “comprising” is intended to mean that the compositions and methods include the recited elements, but do not exclude others. Unless otherwise indicated, open terms for example “contain,” “containing,” “include,” “including,” and the like mean comprising. “Consisting essentially of” when used to define compositions and methods, shall mean excluding other elements of any essential significance to the combination for the intended use. Thus, a composition consisting essentially of the elements as defined herein may not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the present disclosure, which is defined solely by the claims.

The term “about,” as used herein can mean within an acceptable error range for the particular value as determined by one of ordinary skill in the art, which can depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “adenine”, “guanine”, “cytosine”, “thymine”, “uracil” and “hypoxanthine” (the nucleobase in inosine) as used herein generally refer to the nucleobases as such.

The terms “adenosine”, “guanosine”, “cytidine”, “thymidine”, “uridine” and “inosine”, generally refer to the nucleobases linked to the (deoxy)ribosyl sugar.

The term “adeno-associated virus” or “AAV” as used herein generally refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11, sequentially numbered, are disclosed in the prior art. Non-limiting exemplary serotypes useful for the purposes disclosed herein include any of the 11 serotypes, e.g., AAV1, AAV2, AAV3, AAV4, AAV5, AAV6, AAV7, AAV8, AAV9, AAV10, and AAV11.

As used herein the term “amino acid” generally refers to either natural and/or unnatural or synthetic amino acids, including glycine and both the D and L optical isomers, amino acid analogs and peptidomimetics.

The term “deficiency” as used herein can generally refer to lower than normal (physiologically acceptable) levels of a particular agent. In context of a protein, a deficiency can refer to lower than normal levels of the full-length protein.

By a “disorder associated with a nonsense mutation” is generally meant to be a disorder that is caused by a nonsense mutation, and/or having one or more symptoms caused by a nonsense mutation, where the nonsense mutation prevents production of a full-length gene product in an affected cell of the subject. “Disorders associated with a nonsense mutation” encompasses disorders in which a single gene contains one or more nonsense mutations as well as disorders in which two or more (multiple) genes contain one or more nonsense mutations. Exemplary disease or disorders with nonsense mutations include, but are not limited to, cystic fibrosis, beta thalassaemia, Hurler syndrome and Dravet syndrome.

The term “effective amount” generally refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

The term “encode” as it is applied to polynucleotides generally refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are generally used interchangeably when referring to a particular molecule, biological, or cellular material and intend those having minimal homology while still maintaining desired structure or functionality.

As used herein, “expression” generally refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

As used herein, the term “functional” may be generally used to modify any molecule, biological, or cellular material to intend that it accomplishes a particular, specified effect.

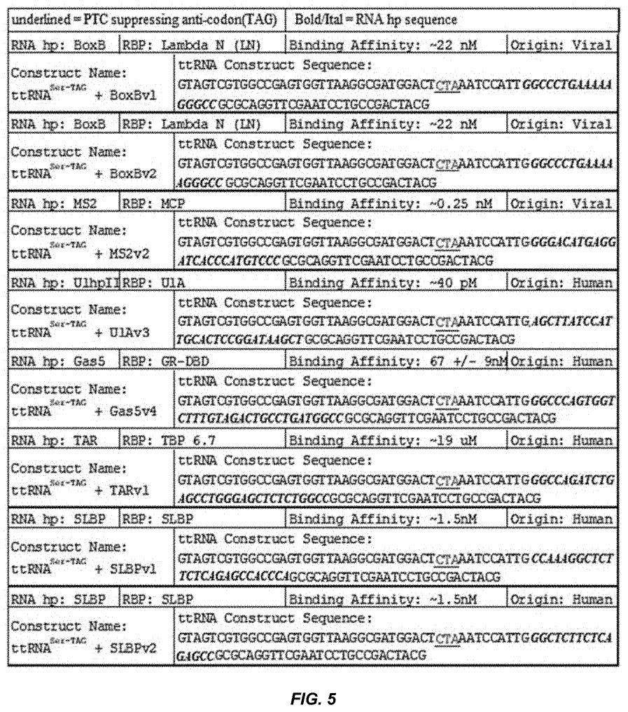

As used herein, the term “fusion protein” generally refers to a protein comprised of domains from more than one naturally occurring or recombinantly produced protein, where generally each domain serves a different function. In one embodiment a fusion protein can comprise two RNA binding proteins linked by a linker domain. In one embodiment, the RNA binding domains are linked such that their respective RNA Recognition Motif (“RRM”) which bind to RNA are functional. In one embodiment, the fusion protein comprises any pair of RNA binding proteins selected from the group consisting of Lambda N (LN), MCP, U1A, GR-DBD, TBP6.7 and SLBP (Table 1). The following table provides the nucleic acid sequences that encode a hairpin motif recognized by the respective RRMs (see also FIG. 5):

TABLE 1

| RNA Binding Protein | Oligonucleotide Motif |
|---|---|
| LN | GGCCCTGAAAAAGGGCC (SEQ ID NO: 21) |
| MCP | GGGACATGAGGATCACCCATGTCCC (SEQ ID NO: 22) |
| U1A | AGCTTATCCATTGCACTCCGGATAAGCT (SEQ ID NO: 23) |
| GR-DBD | GGCCCAGTGGTCTTTGTAGACTGCCTGATGGCC (SEQ ID NO: 24) |
| TBP6.7 | GGCCAGATCTGAGCCTGGGAGCTCTCTGGCC (SEQ ID NO: 25) |
| SLBP | CCAAAGGCTCTTCTCAGAGCCACCCA (SEQ ID NO: 26) |
| SLBP | GGCTCTTCTCAGAGCC (SEQ ID NO: 27) |

The term “gRNA” or “guide RNA” as used herein can generally refer to guide RNA sequences used to target tRNA sequences for gene editing. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. In some aspect, a gRNA is synthetic (Kelley, M. et al. (2016) J of Biotechnology 233 (2016) 74-83).

The terms “hairpin,” “hairpin loop,” “stem loop,” and/or “loop” used alone or in combination with “motif” is used in context of an oligonucleotide to generally refer to a structure formed in single stranded oligonucleotide when sequences within the single strand which are complementary when read in opposite directions base pair to form a region whose conformation resembles a hairpin or loop.

“Homology” or “identity” or “similarity” can generally refer to sequence similarity between two peptides or between two nucleic acid molecules. Homology can be determined by comparing a position in each sequence which can be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the disclosure.

Homology generally refers to a percent (%) identity of a sequence to a reference sequence. As a practical matter, any particular sequence can be at least 50%, 60%, 70%, 80%, 85%, 90%, 92%, 95%, 96%, 97%, 98% or 99% identical to any sequence described herein, which can correspond with a particular nucleic acid sequence described herein or a particular polypeptide sequence. Homology can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

For example, in a specific embodiment the identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity can be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. Similarly, various programs can be used to determine homology/identity between polynucleotide sequences by aligning the sequences in the same orientation (e.g., 5′ to 3′).

“Hybridization” can generally refer to a reaction in which one or more polynucleotides react to form a complex that is stabilized via hydrogen bonding between the bases of nucleotide residues. The hydrogen bonding can occur by Watson-Crick base pairing, Hoogsteen binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Inhibit” as used herein generally refers to the ability to substantially antagonize, prohibit, prevent, restrain, slow, disrupt, alter, eliminate, stop, or reverse the progression or severity of the activity of a particular agent (e.g., infectious agent) or disease.

The term “isolated” as used herein can generally refer to molecules or biologicals or cellular materials being substantially free from other materials. In one instance, the term “isolated” can refer to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also can refer to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and may not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells, or tissues.

The term “lentivirus” as used herein generally refers to a member of the class of viruses associated with this name and belonging to the genus lentivirus, family Retroviridae. While some lentiviruses are known to cause diseases, other lentivirus are known to be suitable for gene delivery. See, e.g., Tomas et al. (2013) Biochemistry, Genetics and Molecular Biology: “Gene Therapy—Tools and Potential Applications,” ISBN 978-953-51-1014-9, DOI: 10.5772/52534.

“Messenger RNA” or “mRNA” is a nucleic acid molecule that is transcribed from DNA and then processed to remove non-coding sections known as introns. The resulting mRNA is exported from the nucleus (or another locus where the DNA is present) and translated into a protein. The term “pre-mRNA” refers to the strand prior to processing to remove non-coding sections.

The term “mutation” as used herein, can generally refer to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those which have no effect on the resulting protein. As used herein the term “point mutation” can refer to a mutation affecting only one nucleotide in a gene sequence. “Splice site mutations” are those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (i.e., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample is benign. As an alternative, absence of a mutation may not indicate that a tissue or sample is benign. Methods as described herein can comprise identifying a presence of a mutation in a sample.

The term “nonsense mutation” as used herein generally refers to a mutation in a nucleic acid sequence that causes premature termination of translation of an messenger RNA into a polypeptide by altering a codon that encodes an amino acid to a sequence encoding a translational termination.

A “nucleic acid affinity tag” generally refers to a moiety or domain on a polynucleotide sequence that can recruit a protein or agent tag. Exemplary protein-binding nucleic acid affinity tags can include, but are not limited to, the MS2 binding sequence, the U1A binding sequence, stem-loop binding protein sequences, the boxB sequence, the eIF4A sequence, or any sequence recognized by an RNA binding protein. A protein or agent tag will interact with a hairpin loop of a polynucleotide sequence. In some embodiments, a guide RNA can include a hairpin loop that recruits a first protein/agent tag. In another embodiment, a ttRNA variable loop can be engineered to comprise a hairpin loop that recruits a second protein/agent tag. In still a further embodiment, the first tag and second tag are linked or can interact with one another. Under this latter embodiment, the two protein/agent tags bring the ttRNA in close proximity to the guide RNA targeting a specific sequence of the mRNA (the target RNA). The guide RNA is typically about 10-30 nucleotides in length. The guide RNA is typically just upstream or downstream of a nonsense mutation to be targeted.

A “pharmaceutically acceptable excipient” generally means an excipient that is useful in preparing a pharmaceutical composition that is generally safe, non-toxic and neither biologically nor otherwise undesirable, and includes an excipient that is acceptable for veterinary use as well as human pharmaceutical use. “A pharmaceutically acceptable excipient” as used in the specification and claims includes both one and more than one such excipient.

The terms “polynucleotide” and “oligonucleotide” are generally used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is generally composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), a nucleoside formed when hypoxanthine is attached to ribofuranose via a β-N9-glycosidic bond, resulting in the chemical structure:

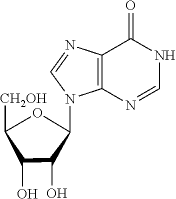

Inosine is read by the translation machinery as guanine (G).

The term “polynucleotide sequence” can be the alphabetical representation of a polynucleotide molecule. This alphabetical representation can be input into databases in a computer having a central processing unit and used for bioinformatics applications such as functional genomics and homology searching. In any alphabetic representation, the disclosure contemplates both RNA and DNA (i.e., wherein “T” is replaced with “U” or vice-a-versa).

The term “protein”, “peptide” and “polypeptide” are generally used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. The term “linker” refers to a protein fragment that is used to link two distinct domains together—optionally to preserve the conformation of the fused protein domains and/or prevent unfavorable interactions between the fused protein domains which may compromise their respective functions. The term “linker” also includes a nucleic acid fragment that separates to distinct domains of a polynucleotide. Typically, a linker in a polynucleotide will maintain a reading frame between domains. In some instances, the linker encodes a “peptide linker”. In some cases, a nucleic acid linker provides for a desired 2 or 3 dimension polynucleotide structure (e.g., the formation of hairpins).

The term “readthrough” as used herein generally refers translation of the sequence of an mRNA without regard to the presence of a premature termination codon (PTC) in the sequence so that translation continues 3′ of the PTC.

As used herein, the term “recombinant expression system” generally refers to a genetic construct or constructs for the expression of certain genetic material formed by recombination; the term “construct” in this regard is interchangeable with the term “vector” as defined herein.

A “recruiting domain” generally includes a polynucleotide sequence that is capable of recruiting or binding with a protein, polypeptide, or peptide (e.g., an RNA binding protein). In some embodiments, a recruiting domain can be a hairpin loop comprising a nucleic acid affinity tag. In certain other embodiments, the recruiting domain is linked to a guide RNA sequence. Exemplary recruiting domains include an Alu domain, an APOBEC domain, a GluR2 domain, Cas13 domain, or any combination thereof. In some case, the Alu domain, APOBEC domain, Cas13 domain, or GluR2 domain can be a naturally occurring recruiting domain. In some cases, the Alu domain, the APOBEC domain, Cas13 domain, or the GluR2 domain can be non-naturally occurring, can be modified from a native sequence, or can be recombinant. At least one of the plurality of recruiting domains can comprise a single stranded sequence. At least one of the plurality of recruiting domains can comprise a plurality of Alu repeats. At least one of the plurality of recruiting domains can form a secondary structure comprising a stem-loop. At least one of the plurality of recruiting domains can form a secondary structure that does not comprise a stem-loop. At least one of the plurality of recruiting domains can form a secondary structure that comprises a cruciform or portion thereof. At least one of the plurality of recruiting domains can form a secondary structure that comprises a toe hold.

As used herein the term “restoring” in relation to expression of a protein can refer to the ability to establish expression of full length protein where previously protein expression was truncated due to mutation. In the context of “restoring activity” the term includes effecting the expression of a protein to its normal, non-mutated levels where a mutation resulted in aberrant expression (e.g., too low or too high).

The term “sample” as used herein, generally refers to any sample of a subject (such as a blood sample or a tissue sample). A sample or portion thereof can comprise a stem cell. A portion of a sample can be enriched for the stem cell. The stem cell can be isolated from the sample. A sample can comprise a tissue, a cell, serum, plasma, exosomes, a bodily fluid, or any combination thereof. A bodily fluid can comprise urine, blood, serum, plasma, saliva, mucus, spinal fluid, tears, semen, bile, amniotic fluid, or any combination thereof. A sample or portion thereof can comprise an extracellular fluid obtained from a subject. A sample or portion thereof can comprise cell-free nucleic acid, DNA or RNA. A sample or portion thereof can be analyzed for a presence or absence or one or more mutations. Genomic data can be obtained from the sample or portion thereof. A sample can be a sample suspected or confirmed of having a disease or condition. A sample can be a sample removed from a subject via a non-invasive technique, a minimally invasive technique, or an invasive technique. A sample or portion thereof can be obtained by a tissue brushing, a swabbing, a tissue biopsy, an excised tissue, a fine needle aspirate, a tissue washing, a cytology specimen, a surgical excision, or any combination thereof. A sample or portion thereof can comprise tissues or cells from a tissue type. For example, a sample can comprise a nasal tissue, a trachea tissue, a lung tissue, a pharynx tissue, a larynx tissue, a bronchus tissue, a pleura tissue, an alveoli tissue, breast tissue, bladder tissue, kidney tissue, liver tissue, colon tissue, thyroid tissue, cervical tissue, prostate tissue, heart tissue, muscle tissue, pancreas tissue, anal tissue, bile duct tissue, a bone tissue, brain tissue, spinal tissue, kidney tissue, uterine tissue, ovarian tissue, endometrial tissue, vaginal tissue, vulvar tissue, uterine tissue, stomach tissue, ocular tissue, sinus tissue, penile tissue, salivary gland tissue, gut tissue, gallbladder tissue, gastrointestinal tissue, bladder tissue, brain tissue, spinal tissue, a blood sample, or any combination thereof.

The term “sequencing” as used herein, can comprise bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, or any combination thereof.

The term “stop codon” intends a three nucleotide contiguous sequence within messenger RNA that signals a termination of translation. Non-limiting examples include in RNA, UAG, UAA, UGA and in DNA TAG, TAA or TGA. Unless otherwise noted, the term also includes nonsense mutations within DNA or RNA that introduce a premature stop codon, causing any resulting protein to be abnormally shortened. tRNA that correspond to the various stop codons are known by specific names: amber (UAG), ochre (UAA), and opal (UGA).

The terms “subject” or “patient” as used herein generally refer to a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. The term “mammal” as used herein refers to a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

The terms “treatment” or “treating” as used herein generally refers to any therapeutic intervention in a subject, usually a mammalian subject, generally a human subject, including: (i) prevention, that is, causing overt clinical symptoms not to develop, e.g., preventing disease progression to a harmful state; (ii) inhibition, that is, arresting the development or further development of clinical symptoms, e.g., mitigating existing clinical symptoms; and/or (iii) relief, that is, causing the regression of clinical symptoms, e.g., causing relief from clinical symptoms.

A “targeting domain” generally refers to a polynucleotide sequence that can be at least partially complementary to a target RNA in a cell. The targeting domain need not be 100% identical to the target RNA, but rather has sufficient identity that it can hybridize to its complementary sequence. A targeting domain includes the complementary RNA antisense sequence to the target RNA as well as DNA sequence that encode (upon transcription) the antisense RNA sequence that is complementary to the RNA target sequence. The targeting domain is typically sufficiently complementary to the target RNA sequence to hybridize under biological condition to the target RNA sequence.

“Transfer ribonucleic acid” or “tRNA” can be a nucleic acid molecule that helps translate mRNA to protein. tRNA have a distinctive folded structure, comprising three hairpin loops; one of these loops comprises a “stem” portion that encodes an anticodon (the anticodon loop). The anticodon recognizes the corresponding codon on the mRNA. Each tRNA is “charged with” an amino acid corresponding to the mRNA codon; this “charging” is accomplished by the enzyme tRNA synthetase. Upon tRNA recognition of the codon corresponding to its anticodon, the tRNA transfers the amino acid with which it is charged to the growing amino acid chain to form a polypeptide or protein. Endogenous tRNA can be charged by endogenous tRNA synthetase. Accordingly, endogenous tRNA are typically charged with canonical amino acids. Orthogonal tRNA, derived from an external source, require a corresponding orthogonal tRNA synthetase. Such orthogonal tRNAs may be charged with both canonical and non-canonical amino acids. In some embodiments, the amino acid with which the tRNA is charged may be detectably labeled to enable detection in vivo. Techniques for labeling are known in the art and include, but are not limited to, click chemistry wherein an azide/alkyne containing unnatural amino acid is added by the orthogonal tRNA/synthetase pair and, thus, can be detected using alkyne/azide comprising fluorophore or other such molecule.

The term “unit dosage form,” as used herein, generally refers to physically discrete units suitable as unitary dosages for human and animal subjects, each unit containing a predetermined quantity of compounds of the present disclosure calculated in an amount sufficient to produce the desired effect in association with a pharmaceutically acceptable excipient (e.g., pharmaceutically acceptable diluent, carrier or vehicle).

The term “vector” generally refers to a polynucleotide (usually DNA) used to artificially carry foreign genetic material to another cell where it can be replicated or expressed. Non-limiting exemplary vectors include plasmids, viral vectors, cosmids, and artificial chromosomes. Such vectors may be derived from a variety of sources, including bacterial and viral sources. A non-limiting exemplary viral source for a plasmid is adeno-associated virus.

DNA is transcribed to RNA, which is translated by ribosomes, causing each amino acid to be linked together one by one to form polypeptides, according to the genetic instructions specifically provided by the DNA. When the ribosome reaches a stop codon, the elongation of the polypeptide terminates. The three stop codons are UAG (amber), UAA (ochre) and UGA (opal). Mutations that occur that change an amino acid-encoding codon to stop codon are called “nonsense mutations.” These nonsense mutations can result in a significant truncation/shortening of the polypeptide sequence, and can cause a profound change in the phenotype of an organism. Thus, even though a gene may be directed to be expressed, a protein encoded by that gene may not be produced because of a nonsense mutation such that when the ribosome reaches the mutant stop signal, it terminates translation resulting in an unfinished protein.

Transfer RNAs translate mRNA into a protein on a ribosome. Each tRNA contains an “anticodon” region that hybridizes with a complementary codon on the mRNA. A tRNA that carries its designated amino acid is called a “charged” tRNA. If the tRNA is one of the 61 amino-acid-associated (i.e., not a stop-signal-associated) tRNAs, it will normally attach its amino acid to the growing polypeptide. The structural gene of tRNA is about 72-90 nucleotides long and folds into a cloverleaf structure. tRNAs are transcribed by RNA polymerase III and contain their own intragenic split promoters that become a part of the mature tRNA coding sequence (Sharp S. J., Schaack J., Coolen L., Burke D. J. and Soil D., “Structure and transcription of eukaryotic tRNA genes”, Crit. Rev. Biochem, 19: 107-144 (1985); Geiduschek E. O., and Tocchini-Valentini, “Transcription by RNA polymerase III, Annu. Rev. Biochem. 57:873-914 (1988)). The stop codon in an mRNA does not pair with the anticodon of a transfer RNA (tRNA), but is recognized by the terminator or release factor, terminating the synthesis of a peptide bond, thereby terminating protein/polypeptide synthesis. Where the termination occurs due to a nonsense mutation, the termination produces an incomplete and typically non-functional protein. The occurrence of nonsense mutations causes premature termination codons (PTC) in the gene box, resulting in two results of gene coding, one that produces truncated proteins and the other that results in the stability of PTC-containing mRNAs.

The nucleotide sequences encoding several hundred human tRNAs are known and are generally available to those of skill in the art through sources such as Genbank (See also Sprinzl et. al., Nucleic Acids Research, volume 12, Supplement “compilation of tRNA Sequences” pp. r1-r57 (1984); Schimmel et. al. Editors, “Transfer-RNA: Structure, Properties, and Recognition, Cold Spring Harbor Labs New York 1979.; Agris, P. F., (1983) “The Modified Nucleosides of Transfer RNA, II, Alan R. Liss Inc., New York (Buckland R A et al., “A cluster of tRNA genes into [DRNI, TRR3, DDRAN] on the short arm of human chromosome 6”, Genomics, 35 164-171 (1996))). The structures of tRNAs are highly conserved and tRNAs are often functional across species. Thus, bacterial or other eukaryotic tRNA sequences are potential sources for oligonucleotides to generate targeting transfer RNAs (ttRNAs) of the disclosure. The determination of whether a particular tRNA sequence is functional in a desired mammalian cell can be ascertained through routine experimentation. Further, additional potential tRNA sequences that are not yet known can be modified as described herein in order to be stabilized through routine experimentation. tRNA genes have strong promoters that are active in all cell types. The promoters for eukaryotic tRNA genes are contained within the structural sequences encoding the tRNA molecule itself. Although there are elements that regulate transcriptional activity within the 5′ upstream region, the length of an active transcriptional unit may be considerably less than 500 base pairs and thus accommodation within a delivery vector is straightforward. Once they have been transcribed and processed, tRNAs have low rates of degradation.

The disclosure provides ttRNAs that generally produce a tRNA cloverleaf-like structure. The ttRNAs are derived from a core tRNA sequence specific for a codon, wherein the anticodon loop has been modified to interact with a stop codon in an mRNA and wherein the variable loop has been modified to interact with a cognate-pair of a protein-pair or with RNA binding protein (“tRNA targeting domain”). As mentioned above, sequences for tRNAs are known and can be readily searched and obtained on the world-wide-web (see, e.g., [http://]trna.ucsc.edu/tRNAviz/). The following consensus sequence for tRNAs in primates are provided in Table 2.

TABLE 2
Consensus Sequences of tRNAs

| Sequence: B = C/G/U; D = A/G/U; H = A/C/U; K = G/U; M= A/C; R = PURINE | |
|---|---|
| CONSENSUS; S = C/G; W = A/U; x = ABSENT or variable; | |
| Y = PYRIMIDINE; z = PAIRED CONSENSUS (e.g., A/T, A/U or G/C); | Isotype |
| GGGGRWKUAGCUC [symbol] GAGCGCzYzCUUHGCAUGzRzGAGxxxxxxxxxxx | Ala |
| xxxxXXXXGYMSYGGGWUCRAYSCCCRSMWYCUCCA (SEQ ID NO: 28) | |
| ZzSYzzGUGGCSx [symbol] xSGCRzYKzMCUHCKRAKzMRzRGxxxxxxxxxxx | Arg |
| xxxxxxxxRUUzzRGGUUCGAVUCCYzzCzzRSzzR (SEQ ID NO: 29) | |
| ZUCzCUGUGGCzC [symbol] GzGCGUzzzGCUGUUAACzzzAAGxxxxxxxxxxx | Asn |
| xxxxxxxxGUUGGUGGUUCRADCCCACCCAGzGAzG (SEQ ID NO: 30) | |
| UGGzCZUUAGUAU [symbol] GUAUYCCCzCCUGUCACGzGGGAGxxxxxxxxxxx | Asp |
| xxxxxxxxAxCCSGGGUUCRAUUCCCSGAzGzGGAG (SEQ ID NO: 31) | |
| GGGGGUzUAGCUC [symbol] GAGCAUUUGACUGCAGAUCAAGAGxxxxxxxxxxx | Cys |
| xxxxxxxxGUCCCzRGUUCAAAUCYzGGzGCCCCCU (SEQ ID NO: 32) | |
| GGxYCCAUGGUGU [symbol] GCACUCUGGACUYUGAAUCCAGYRxxxxxxxxxxx | Gin |
| xxxxxxxxAXUCCGAGUUCRARUCUCGGUGGRxCCU (SEQ ID NO: 33) | |
| UCCCWzzURGUCU [symbol] GGAUUCzKSGYUYUCACCSMzGYGxxxxxxxxxxx | Glu |
| xxxxxxxxRxCCCGGGUUCRAYUCCCGGzzWGGGAA (SEQ ID NO: 34) | |
| GCRYzGSUGGUxx [symbol] xxAUDzzzzMYUBCCAHKzzzzHGxxxxxxxxxxx | Gly |
| xxxxxxxxRxCCzRGGUUCRAUUCCYzGSCzRYGCA (SEQ ID NO: 35) | |
| GYCxUKAUCGUAU [symbol] GUACUYUGCGUUGUGGCCGCARCAxxxxxxxxxxx | His |
| xxxxxxxxAxCCUCGGUUCRAAUCCGAGUMAxGRCA (SEQ ID NO: 36) | |
| GSYCxRKURGCKC [symbol] GMGCRYGGzRCUDAUAAYzCCRAGxxxxxxxxxxx | He |
| xxxxxxxxGUYzYGRGUUCRABYCYCRzMYxGRSCA (SEQ ID NO: 37) | |
| RzzRGzRUGGCCx [symbol] xGGCGYYRSRYUHARGHYSYRRUxSzzxxxxxxxx | Leu |
| xxxxxzzSGxCGUGGGUUCGAAYCCCACYzCYzzYA (SEQ ID NO: 38) | |
| ZCCzzzUAGUC [symbol] GAGCAUSRGACUYUUAAUCYSAGGxxxxxxxxxxxxx | Lys |
| xxxxxxGUCxWGGGUUCRAGYCCCWxzzzzGGzR (SEQ ID NO: 39) | |
| GCCzYSUUAGCGC [symbol] GCGCGUCAGxCUCAUAAxCUGAAGxxxxxxxxxxx | Met |
| xxxxxxxxGUCSUGAGUUCRADCCUCASASRzGGCA (SEQ ID NO: 40) | |
| GCYzAAAUAGCUC [symbol] GAGCRUUAGACUGAAGAUCUAAAGxxxxxxxxxxx | Phe |
| xxxxxxxxGUCCCUGGUUCRAUCCCGGGUUUzRGCA (SEQ ID NO: 41) | |
| GGCUCGUUGGUCx [symbol] xGAUUCUCGSUUHGGGUSCGAGAGxxxxxxxxxxx | Pro |
| xxxxxxxxGUCCCGGGUUCAAAUCCCGGACGAGCCC (SEQ ID NO: 42) | |
| GzzRzSRUGzCCx [symbol] xGzGWUGGACUxSWAAUCCAWUxGKGSxxxUYUxx | Ser |
| xxxSCMCGxCzYRGGUUCGAAUCCYRzYSzYzzCG (SEQ ID NO: 43) | |
| GGCzCzzURGCYx [symbol] xRGCRYYKGUCUHGUAAACMRRRGxxxxxxxxxxx | Thr |
| xxxxxxxxRUCZZGRGUUCRAHYCYCzzzzGzGCCU (SEQ ID NO: 44) | |
| GzCCzCGUGGCGC [symbol] GCGCGUCUGACUCCAGAUCAGAAGxxxxxxxxxxx | Trp |
| xxxxxxxxGYUGCGUGUUCRARUCACGUCGzGGzCA (SEQ ID NO: 45) | |
| CCUzCGAUAGCUC [symbol] GAGCGGAGGACUGUAGAUCCUUAGxxxxxxxxxxx | Tyr |
| xxxxxxxxGUCGCUGGUUCGAWUCCGGCUCGzAGGA (SEQ ID NO: 46) | |
| GKUzCzRUAGUGx [symbol] xCACRUYYGCYUHACACGCRRAAGxxxxxxxxxxx | Val |
| xxxxxxxxGUCCYzGGKUCGADMCCzRGYzGzAMCA (SEQ ID NO: 47) | |

bold + ital = D-Loop; Underline = anticodon loop; Ital = variable loop; bold = T-Loop; (*)
*″U″ can be replaced with ″T″ to arrive at DNA sequences in the table above.

Using, for example, the tRNA sequences available in the art, or as presented above in Table 2, one can modify the anticodon loop (the underlined sequence above) to target a stop codon in a nonsense mutation. Accordingly, the disclosure provides a polynucleotide comprising a tRNA sequence wherein the anticodon loop has been mutated to bind to a stop codon sequence in an mRNA and transfer its charged amino acid to the growing protein chain.

In one embodiment, the disclosure thus provides a polynucleotide encoding for a tRNA cloverleaf structure, wherein the anticodon loop of the tRNA cloverleaf is from 5′->3′: N₁UCTAN₂N₃ (SEQ ID NO:18); N₁UUUAN₂N₃ (SEQ ID NO:19); or N₁UUCAN₂N₃ (SEQ ID NO:20), wherein N₁ is a pyrimidine, N₂ is a purine and N₃ is any of U, A or C.

The tRNA cloverleaf sequence can be modified at the variable domain (the italicized domain above) to contain a sequence that interacts with an RNA binding domain or a first RNA binding protein of a fusion construct of two RNA binding proteins.

The variable loop of the ttRNA is modified such that it comprises a sequence with is recognized by a protein/agent tag (e.g., “tRNA targeting domain”). The variable loop domain of the ttRNA can be any sequence recognized by an RNA binding protein or other affinity tag. Numerous such sequences are known. Nucleic acid affinity tags can include, for example, a chemical tag, an RNA-binding protein binding sequence, a DNA-binding protein binding sequence, a sequence hybridizable to an affinity-tagged polynucleotide, a synthetic RNA aptamer, or a synthetic DNA aptamer. Examples of chemical nucleic acid affinity tags can include, but are not limited to, ribo-nucleotriphosphates containing biotin, fluorescent dyes, and digoxigenin. Examples of protein-binding nucleic acid affinity tags can include, but are not limited to, the MS2 binding sequence, the U1A binding sequence, stem-loop binding protein sequences, the boxB sequence, the eIF4A sequence, or any sequence recognized by an RNA binding protein. Examples of nucleic acid affinity-tagged oligonucleotides can include, but are not limited to, biotinylated oligonucleotides, 2, 4-dinitrophenyl oligonucleotides, fluorescein oligonucleotides, and primary amine-conjugated oligonucleotides.

For example, one sequence recognized by RNA binding domain (e.g., BoxB) is located in the variable domain of the ttRNA. In a particular embodiment, the sequence recognized by the RNA binding domain is a BoxB sequence. In a particular embodiment, the BoxB sequence comprises GCCCUGAAAAAGGGC (SEQ ID NO: 14) or GGCCCUGAAAAAGGGCC (SEQ ID NO: 15). In a particular embodiment, the BoxB sequence has at least 80%, 85%, 90%, 95%, 97%, 99%, or 100% homology or identity, particularly at least 95%, 97%, 99%, or 100% homology or identity, to SEQ ID NO: 14 or 15. In some embodiments, the sequence recognized by an RNA binding domain is selected from the group of an MS2 stem loop and a BoxB loop and/or are stabilized by replacing A-U with G-C. In another embodiment, the sequence is a sequence that is bound by Csy4 and comprises GTTCACTGCCGTATAGGCAG (truncated 20 nt) (SEQ ID NO: 16) or GUUCACUGCCGUAUAGGCAGCUAAGAAA (SEQ ID NO:17).

The disclosure provides a targeting transfer RNA (ttRNA) comprising a polynucleotide having a general structure from 5′ to 3′ of: 5′-(D-Loop domain)-(anticodon Loop domain)-(variable loop domain)-(T loop domain)-3′, wherein the anticodon loop domain comprises a sequence selected from N₁UCTAN₂N₃ (SEQ ID NO:18); N₁UUUAN₂N₃ (SEQ ID NO:19); or N₁UUCAN₂N₃ (SEQ ID NO:20), wherein N₁ is a pyrimidine, N₂ is a purine and N₃ is any of U, A or C, that binds to a stop codon sequence of an mRNA; wherein the variable loop domain is about 12-30 nucleotides in length of a nucleic acid affinity tag and comprises a sequence that binds to a protein or agent tag, and wherein the ttRNA suppresses nonsense mutations in messenger RNA. In one embodiment, the ttRNA comprise a nucleic acid sequence that binds with an RNA binding protein. In a further embodiment, the nucleic acid sequence is present in the variable loop domain. In another embodiment, the ttRNA is derived from a tRNA, wherein the tRNA is engineered to contain an anticodon loop that binds to a stop codon of an mRNA. In a further embodiment, the tRNA is engineered such that the variable loop domain is replaced or engineered to contain a nucleic acid affinity tag sequence that is recognized by an RNA binding protein. In still another embodiment, the tRNA that is engineered can be a tRNA for Ala, Arg, Asn, Asp, Cys, Gln, Glu, Gly, His, Ile, Leu, Lys, Met, Phe, Pro, Ser, Thr, Trp, Tyr, and Val.

In a further embodiment, the disclosure can include a guide RNA comprising at least one domain comprising a nucleic acid affinity tag sequence that interacts with an RNA binding protein and a sequence that is complementary to a target region of an mRNA upstream or downstream of a PTC. In a further embodiment, the guide RNA is targeted upstream or downstream of a nonsense mutation such that the guide RNA interacts with the mRNA allowing for an RNA binding protein to bind to the at least one nucleic acid affinity tag sequence, wherein a ttRNA can be targeted to the guide RNA location by the modified variable loop domain that binds to a RNA binding protein or agent linked to the RNA binding protein bound by the gRNA-affinity tag sequence.

Nonsense mutations, also called Premature Termination Codons (PTCs), make up 10-15% of the single base pair mutations that cause human disease. Cystic fibrosis is one such disease that results from PTCs (Peltz et al., Annu Rev Med., 64:407-25, 2013). In general, nonsense mutations have more serious ramifications than missense mutations because of the almost complete loss of gene expression and activity and with the possibility of dominant negative effects of truncated products. PTCs result in premature translation termination and accelerated mRNA transcript decay through the Nonsense Mediated Decay (NMD) pathway.

The ability to suppress premature termination codons (PTCs) via engineered ttRNAs is an extremely powerful and versatile tool with a multitude of clinical opportunities. It has been found that certain amino acid codons (one nucleotide change away from a stop codon) are the most vulnerable to nonsense mutations, and that several versions of each human tRNA are associated with these amino acids. The native anticodon loop of the tRNA can be converted to one that would bind to a PTC by using mutagenesis.

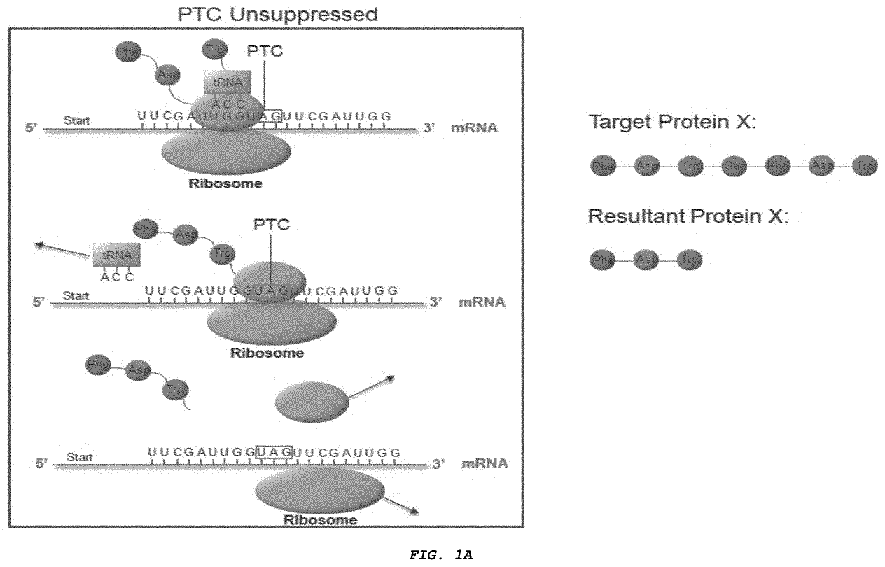

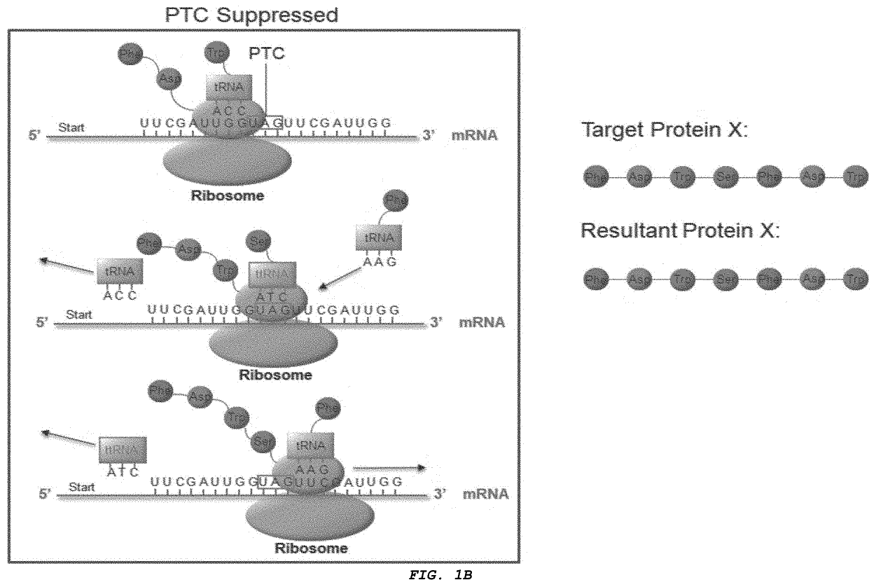

In various embodiments presented herein, the disclosure provides for methods for treating these diseases by reversing the effects of PTCs through introduction of the ttRNAs of the disclosure. FIG. 1 provides a schematic of how the PTC suppressor ttRNA system works in rescuing translation. This approach allows for the engineering of a host of treatments for a wide variety of PTC associated diseases, like cystic fibrosis and Duchenne muscular dystrophy. One important benefit of the ttRNA approach, is that rescued protein expression is temporary and offers an alternative to more permanent genetic therapies such as CRISPR-Cas9.

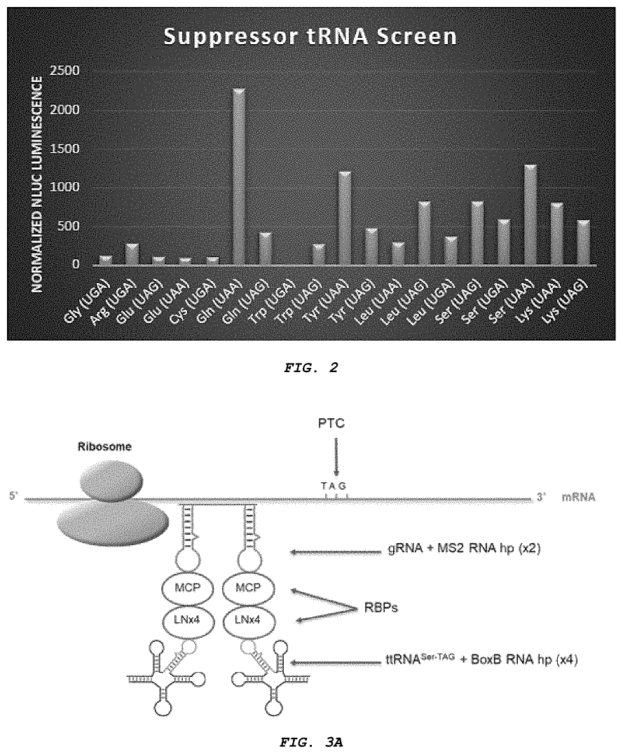

The disclosure demonstrates this using luciferase reporter, wherein a luciferase reporter is modified with a PTC associated with each ttRNA. Luminescence results only if the ttRNA rescued expression of the reporter by suppressing the PTC. The signal recovery for each ttRNA and PTC type is plotted in FIG. 2. Important to note, although the screen focuses on tRNAs associated with amino acids most vulnerable to nonsense mutations, this approach is possible for any tRNA. The results of the luciferase screen highlight the potential of the ttRNA approach to suppress PTCs. Excluding Trp (UGA), each of these tRNA suppressors achieved a minimum ˜100-fold increase over background. Notably, some suppressors such as Gln (UAA) had signals >2200-fold over background.

Preliminary in vitro and in vivo work has been conducted using the ttRNA approach. Research has demonstrated rescue of production of cystic fibrosis transmembrane conductance regulator (CFTR) in HEK293 cells. CFTR is a large transmembrane protein that controls anion transport in many tissues and organs. Nonsense mutations can disrupt the production of this protein leading to cystic fibrosis (CF). In the studies, HEK293 cells were transfected with CFTR (modified with a UGA PTC) and the associated suppressor tRNA. Using western blots, some protein recovery was observed in vitro. In vivo activity of this approach was examined in mice. A nLucifersase (nLuc) reporter (modified with a UGA PTC), along with a plasmid containing four copies of the tRNA suppressor (Arg in this case) was delivered into mouse muscle tissue using electroporation. nLuc luminescence was measured at 1, 2 and 7 days. The experiments measured nLuc levels that were comparable to the WT levels, showing this approach can work in vivo as well.

As described herein, the ttRNAs of the disclosure suppress PTCs that result from nonsense mutations. As such, this type of system falls into the category of genetic therapy, and is a feasible approach for any disease that results from nonsense mutations. According to statistics, about 11.2% of hereditary diseases produce PTC mutations, called premature termination codons diseases (PTC diseases). On the other hand, many cancers also produce PTC mutations. For example, Atkinson and Martin in 1994 identified close to 180 unique point mutations to nonsense codons identified in human genes from a search of literature reports. These types of mutations result in Duchenne muscular dystrophy, Xeroderma pigmentosum, cystic fibrosis, hemophilia, anemia, hypothyroidism, p53 squamous cell carcinoma, p53 hepatocellular carcinoma, p53 ovarian carcinoma, esophageal carcinoma, osteocarcinoma, ovarian carcinoma, esophageal carcinoma, hepatocellular carcinoma, breast cancer, hepatocellular carcinoma, fibrous histiocytoma, ovarian carcinoma, SRY sex reversal, triosephosphate isomerase-anemia, diabetes and rickets. The BRCA-1 and BRCA-2 genes associated with breast cancer also have similar mutations which may be treated with the ttRNAs disclosed herein.

Duchenne muscular dystrophy (DMD) is a typical representative of PTC-associated disease. DMD is a serious muscle atrophy disease and the most common X-linked recessive hereditary disease. It is characterized by progressive debilitation and, ultimately, lethality. A nonsense mutation is one of the main causes of DMD. The nonsense mutation produces a premature stop codon UAG, UAA, UGA, resulting in a truncated polypeptide product that causes the patient to lack functional dystrophin, resulting in muscle atrophy. According to reports, the incidence of Duchenne muscular dystrophy in live births is 1/6300 to 1/3500. 40% of these mutations manifest as nonsense mutations that result in a truncated form of the Dystrophin protein. There is no effective cure for this disease. The disease is identified in the early onset of childhood, loss of walking ability in adolescence, death in early adulthood, and a heavy psychological and economic burden on patients, families and society. Dystrophin serves as an anchor between the cytoskeleton and cell membrane in muscular tissue. Patients suffering from DMD often lack Dystrophin, and their muscle cells suffer damage during contractions and eventually die off. Over time the accumulated cell death results in muscle atrophy which can be fatal.

The compositions, and methods disclosed herein offer viable alternatives to more permanent genetic therapies, such as CRISPR-Cas9. Although CRISPR approaches are quite powerful, there is some hesitance in its use in the clinic due to technical and ethical concerns. A portion of this hesitance is derived from the permanency of the approach. On the other hand, RNA based approaches (such as those described herein) are temporary in nature.

The ttRNAs disclosed herein and the sequences encoding them or functional equivalents thereof can be used for any of a number of genetic engineering protocols. In one embodiment, sequences encoding ttRNAs are introduced into a cell in a gene therapy protocol whether in vitro, ex vivo, or in vivo to suppress the effects of mutations which result in truncated and inactive gene products responsible for disease. The ttRNA encoding oligonucleotides can be directly introduced to cells. They are so similar to native tRNAs such that they will be unlikely to generate significant immune response. Additionally, and in another embodiment for increased delivery of ttRNA, oligonucleotide sequences for ttRNAs may be contained within an appropriate expression vehicle comprising a nucleotide vector.

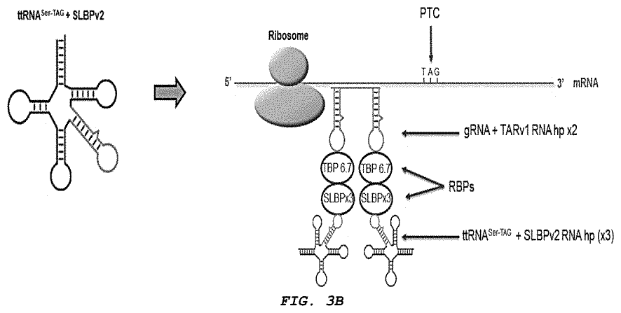

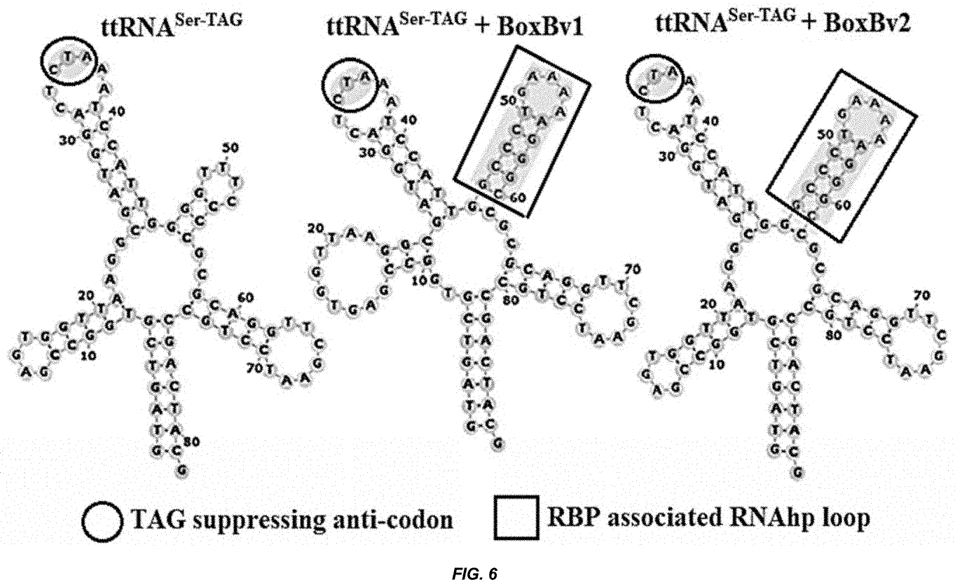

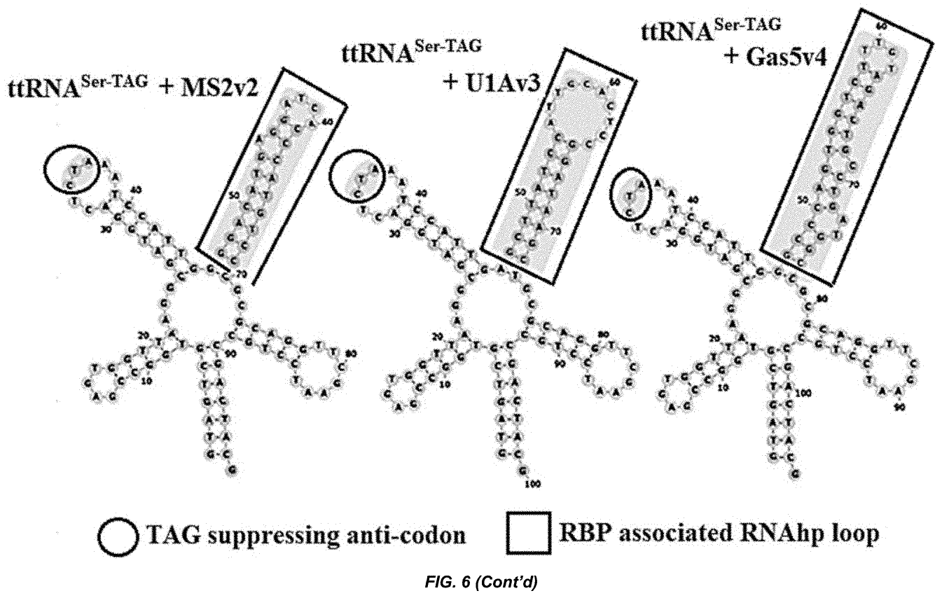

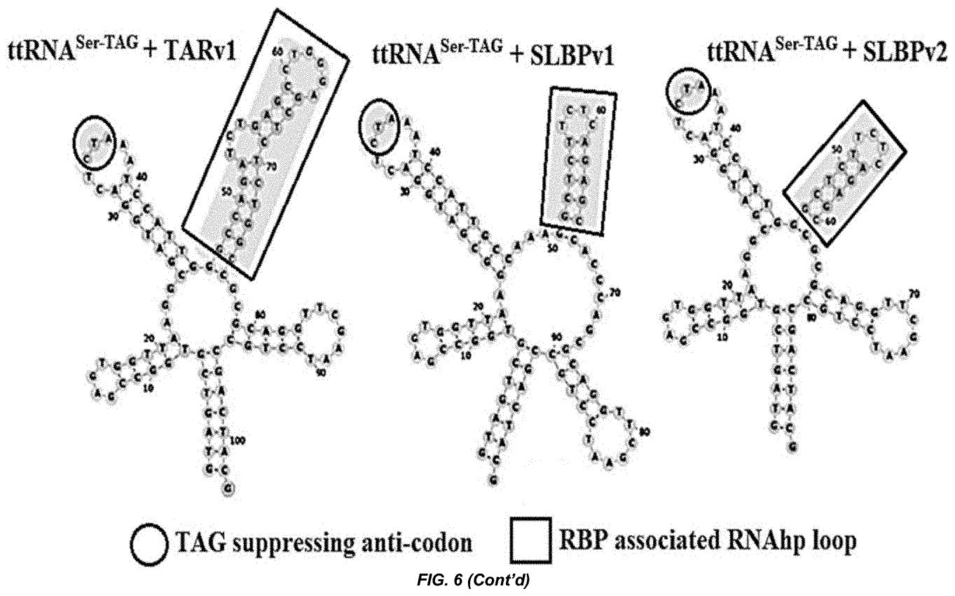

The ttRNAs of the disclosure allow for “re-editing” of a disease-causing nonsense codon to a specific amino acid. The ttRNAs target only one type of stop codon, such as, e.g., TGA (UGA) over TAC or TAA. The small size of the ttRNA molecules makes them amenable for ready expression, as the ttRNA with a typical promoter is generally less than 500 bp. Briefly, an oligonucleotide is synthesized that comprises the structural component of a tRNA gene functional in human cells with modifications made in the anticodon region of the tRNA, and wherein the variable loop of the tRNA is replaced with a nucleic acid affinity tag (e.g., known RNA aptamers) that have strong binding affinity to specific RNA binding proteins. Thus, the ttRNAs of the disclosure recognize a PTC and further can be used in systems, such as described in FIG. 3A-B, to increase efficiency and specificity of PTC suppression by use RNA binding protein complexes to target specific sequences. The tRNA gene that is functional in human cells can be selected from any of the 61 types of tRNAs that have a one-to-one correspondence with the 61 sense codons of the standard genetic code. In particular embodiment, the tRNA gene is from tRNA genes that have one-to-one correspondence with sense codons for serine or arginine. The ttRNAs can comprise anticodons for any of the three different stop codons (DNA sequence/RNA sequence): TAG/UAG (“amber”); TAA/UAA (“ochre”); and TGA/UGA (“opal”). In a particular embodiment, the ttRNA comprises an anticodon that recognizes the TAG stop codon. In yet a further embodiment, the ttRNA is ttRNA^{Ser-TAG} that further comprises an RNA aptamer that has strong binding affinity to a specific RNA binding proteins. Examples of such ttRNA and RNA aptamers include those presented in FIG. 6, namely BoxBv1, BoxBv2, MS2v2, U1Av3, Gas5v4, TARv1, SLBPv1, and SLBPv2. In a further embodiment, the ttRNA is ttRNA^{Ser-TAG} with a BoxBv1 RNA aptamer (nucleic acid affinity tag).

The ttRNAs, or polynucleotide sequences encoding such ttRNAs, can be introduced to cells to suppress PTCs. The ttRNAs can be introduced into recipient cells using standard conventional genetic engineering techniques, e.g., through use of vectors or by transfection. In a particular embodiment, an appropriate gene transfer vehicle is used to transduce cells to express the ttRNA. The gene delivery vehicle can be any delivery vehicle known in the art and can include naked DNA which is facilitated by a receptor mediated transfection as well as any of a number of vectors. Such vectors include, but are not limited to, eukaryotic vectors, prokaryotic vectors (such as for example bacterial vectors) and viral vectors including but not limited to retroviral vectors, adenoviral vectors, adeno-associated viral vectors, alphaviruses, lentivirus vectors (human and other including porcine), Herpes virus vectors, Epstein-Barr virus vectors, SV40 virus vectors, pox virus vectors, pseudotype virus vectors and gammaretroviruses (e.g., MLV).

In a particular embodiment, the viral vector is a retroviral or an adeno-associated viral vector. Examples of retroviral vectors which may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus.

Retroviral vectors are useful as agents to mediate retroviral-mediated gene transfer into eukaryotic cells. Retroviral vectors are generally constructed such that the majority of sequences coding for the structural genes of the virus are deleted and replaced by the gene(s) of interest. Most often, the structural genes (i.e., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal.

These genes are incorporated into the proviral backbone in several general ways. The most straightforward constructions are ones in which the structural genes of the retrovirus are replaced by a single gene which then is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

In a particular embodiment, the viral vector is an adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficient and selectivity, e.g., biotinylated AAV vectors, directed molecular evolution, self-complementary AAV genomes and so on. Modified AAV have also been described, including AAV based on ancestral sequences; see, e.g., U.S. Pat. No. 7,906,111; WO/2005/033321; WO2008027084, WO2014124282; WO2015054653; and WO2007127264. Other modified AAVs that have been described include chimeric nanoparticles (ChNPs) that have an AAV core that expresses a transgene that is surrounded by layer(s) of acid labile polymers that have embedded antisense oligonucleotides (e.g., see Hong et al., ACS Nano 10:8705-8716 (2016)) and Cho et al., Biomaterials 2012, 33, 3316-3323). The compositions and methods disclosed herein is a platform technology, and as such the composition and methods disclosed herein can be used with all known AAVs, including the modified AAVs described in the literature, such as ChNPs.

In another embodiment, the viral vector is an adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68: 143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986). Alphaviruses can also be used. Alphaviruses are enveloped single stranded RNA viruses that have a broad host range, and when used in viral gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1(1): 13-25; Lundstrom, Viruses. 2014 June; 6(6): 2392-2415; Lundstrom, Curr Gene Ther. 2001 May; 1(1): 19-29; Rayner et al., Rev Med Virol. 2002 September-October; 12(5):279-96.

The disclosure further provides for pharmaceutical compositions and formulations comprising a ttRNA (or a vector encoding a ttRNA) for specified modes of administration and delivery of a ttRNA as described herein. In one embodiment, a ttRNA (or an oligonucleotide encoding a ttRNA) described herein is an active ingredient in a composition comprising a pharmaceutically acceptable carrier. Such a composition is referred to herein as a pharmaceutical composition. A “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions. When an agent, formulation or pharmaceutical composition described herein, is administered to a subject, preferably, a therapeutically effective amount is administered. As used herein, the term “therapeutically effective amount” refers to an amount that results in an improvement or remediation of the condition.

Administration of the pharmaceutical composition to a subject is by methods that allow the ttRNA (or a vector encoding a ttRNA) to be delivered to a target cell or tissue. The specific route will depend upon certain variables such as the target cell and can be determined by the skilled practitioner. Suitable methods of administering a ttRNA (or a vector encoding a ttRNA) described herein to a patient include any route of in vivo administration that is suitable for delivering ttRNA (or a vector encoding a ttRNA) to a patient. Various routes of administration will be apparent to those of skill in the art, depending on the preparation's type of viral gene therapy being used, the target cell population, and the disease or condition experienced by the subject. Methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In an embodiment where the target cells are in or near a tumor, one route of administration is by direct injection into the tumor or tissue surrounding the tumor. For example, when the tumor is a breast tumor, the methods of administration include impregnation of a catheter, and direct injection into the tumor.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189: 11277-11281, 1992, which is incorporated herein by reference in its entirety). Oral delivery can be performed by complexing ttRNA (or a vector encoding a ttRNA) to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering the ttRNA (or a vector encoding a ttRNA) to a cell or tissue that is accessible by surgery, and particularly, on or near the surface of the body. Administration of a composition locally within the area of a target cell refers to injecting the composition centimeters and preferably, millimeters from the target cell or tissue.

The appropriate dosage and treatment regimen for the methods of treatment described herein will vary with respect to the particular disease being treated, the ttRNA (or a vector encoding a ttRNA) being delivered, and the specific condition of the subject. The skilled practitioner is to determine the amounts and frequency of administration on a case by case basis. In one embodiment, the administration is over a period of time until the desired effect (e.g., reduction in symptoms is achieved). In a certain embodiment, administration is 1, 2, 3, 4, 5, 6, or 7 times per week. In a particular embodiment, administration is over a period of 1, 2, 3, 4, 5, 6, 7, 8, 9, or 10 weeks. In another embodiment, administration is over a period of 2, 3, 4, 5, 6 or more months. In yet another embodiment, treatment is resumed following a period of remission.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more ttRNAs (or vectors encoding ttRNAs) described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

The disclosure further provides methods for restoring translation in a cell of a nucleotide sequence that has a PTC comprising contacting the cell with a ttRNA or vector encoding a ttRNA disclosed herein. In a particular embodiment, the cell is contacted in vitro. In an alternate embodiment, the cell is contacted in vivo or ex vivo.

The disclosure also provides methods for treating a subject that has a disorder associated with a nonsense mutation, comprising administering to the subject an effective amount of a ttRNA disclosed herein or a vector encoding a ttRNA disclosed herein. In a further embodiment, the vector encoding the ttRNA is a viral vector. In yet a further embodiment, the viral vector is a retroviral vector or adeno-associated viral vector.

The disclosure further provides that the ttRNAs disclosed herein can be used in combination with specifically engineered guide RNAs (gRNAs) and/or RNA binding protein fusion constructs to allow for localization of the ttRNA to specific mRNAs of interest, thereby minimizing off target suppression of native stop codons in vicinity of the 3′ UTR, and increasing the efficiency of PTC suppression in general.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### Examples

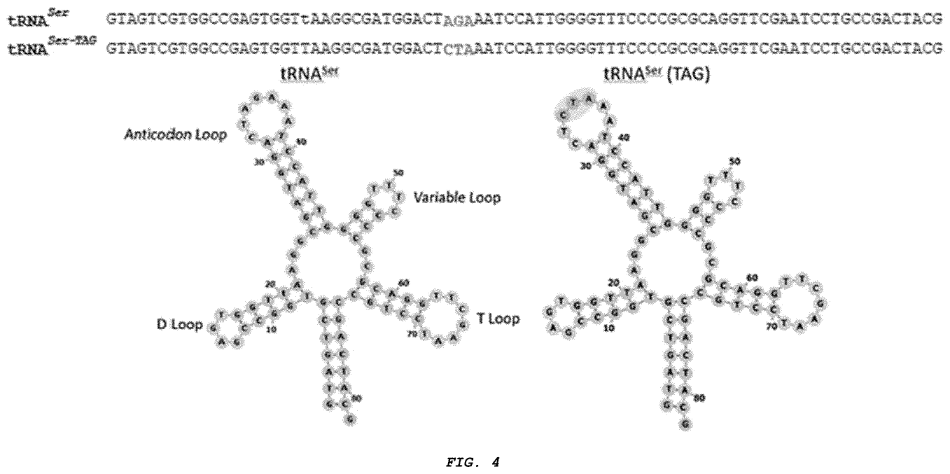

ttRNA selection/engineering for PTC suppression. A suitable tRNA to perform the studies presented herein was selected, ttRNA^{Ser}. The tRNA for Serine has two notable advantages: First, ttRNA^{Ser} has a well-defined variable loop that when, replaced with RNA hairpins, did not significantly impact the overall secondary structure of the tRNA. Similarly, it accepted modifications to the anticodon loop with no structural changes; Second, TAG was the most advantageous termination codon for the studies. ttRNA^{Ser-TAG} had the very high signal recovery (e.g., see FIG. 2). FIG. 4 shows the tRNA sequence that was selected and modified. The figure further provides visualization of the modifications to the anticodon loop. This process can be applied to tRNA of any amino acid isotype and for any of the three stop codons. As such PTCs of various diseases can be specifically targeted using the processes disclosed herein. Additionally, the specific ttRNA can be used to replace the native amino acid missing from the protein of interest.

ttRNA engineering for increased efficiency/localization/specificity. The next step of the ttRNA design process was to build in the ability to target specific mRNAs and stop codons of interest. The idea is to minimize off target suppression of native stop codons and to localize the ttRNA to the target mRNA in order to increase efficiency. The approach used herein was to replace the variable loop of the ttRNA with known RNA hairpin aptamers. These aptamers are RNA hairpin loops that bind to specific RNA binding proteins. Finally, the use of gRNAs specific to the mRNA of interest will allow the localization of the RNA binding proteins and the ttRNA+RNA hairpin constructs in close proximity to the PTC to be suppress. The schematic shown in FIG. 3 is an example of how this process will work with known RNA hairpins and associated RNA binding proteins. The ribosomal machinery is robust enough to free the system (guide RNA-ttRNA complex) from the mRNA of interest. The combination of proximity and superior binding affinity of the ttRNA to the ribosome will increase efficiency and specificity of PTC suppression.

RNA hairpins and their associated RNA binding proteins were tested in the studies to assess the following criteria. First, can the particular ttRNA's variable loop region support the structure of the RNA hairpin without severe conformational changes. Studies have shown that certain stem sequences can affect the overall conformation of the ttRNA. Second, does the physical size of the RNA hairpin interfere with the ttRNA's ability to interact with the ribosome for suppression or the synthetase for amino acylation. Third, analysis of the binding affinity of the RNA hairpin to RNA binding proteins compared with ttRNA to ribosome. If the binding affinity is too high, the ttRNA may not release from the complex and could be inhibitory to the interaction with the ribosome. If the binding affinity is too low, the ttRNA+RNA hairpin may not efficiently localize to the mRNA of interest. Finally, are the RNA hairpins associated with endogenous and human RNA binding proteins. Ideally human based RNA hairpins would provide for the recruitment of endogenous RNA binding proteins, allowing for the packaging and delivery of less cargo and avoiding immunogenic effects. FIG. 5, provides a table of some of the ttRNA+RNA hairpin constructs that were tested with associated sequences. FIG. 6 provides visualizations of the constructs listed in FIG. 5.

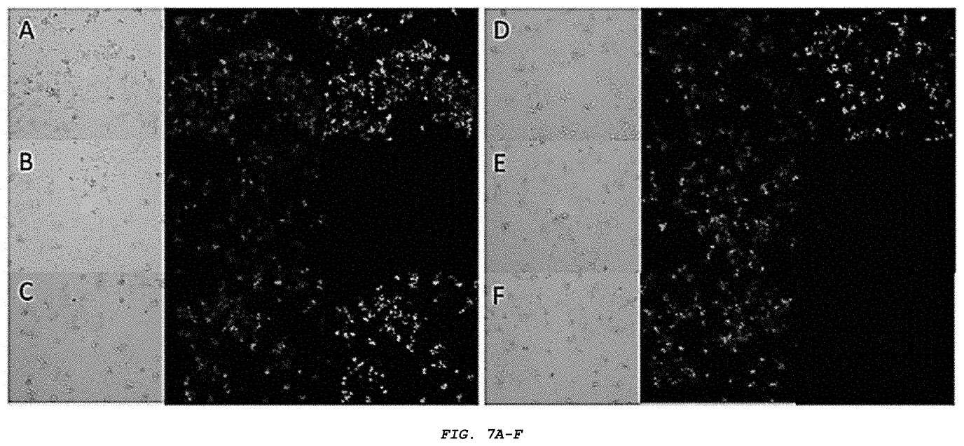

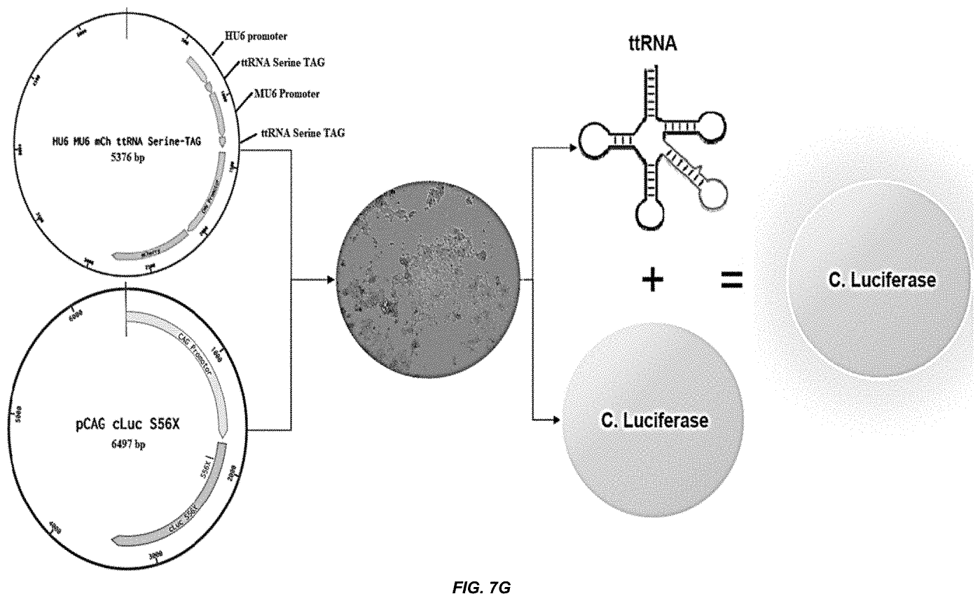

A simple assay was used to assess if PTC suppression functionality of the ttRNA constructs were maintained after modifying the variable loop with RNA hairpin. The ttRNA constructs were first cloned into separate HU6/MU6 plasmids with a U6 promotor. Additionally, a mCherry reporter was included to serve as an internal control. A GFP reporter (modified with a TAG PTC) was also cloned into a HU6/MU6 plasmid as well. The positive control was a HU6/MU6 GFP plasmid with no PTC. The negative control was the HU6/MU6 GFP-TAG plasmid lacking the ttRNA suppressor. HEK293 cells were transfected with the aforementioned plasmids in a 24 well plate format. FIG. 7A-F shows the experimental design and results.

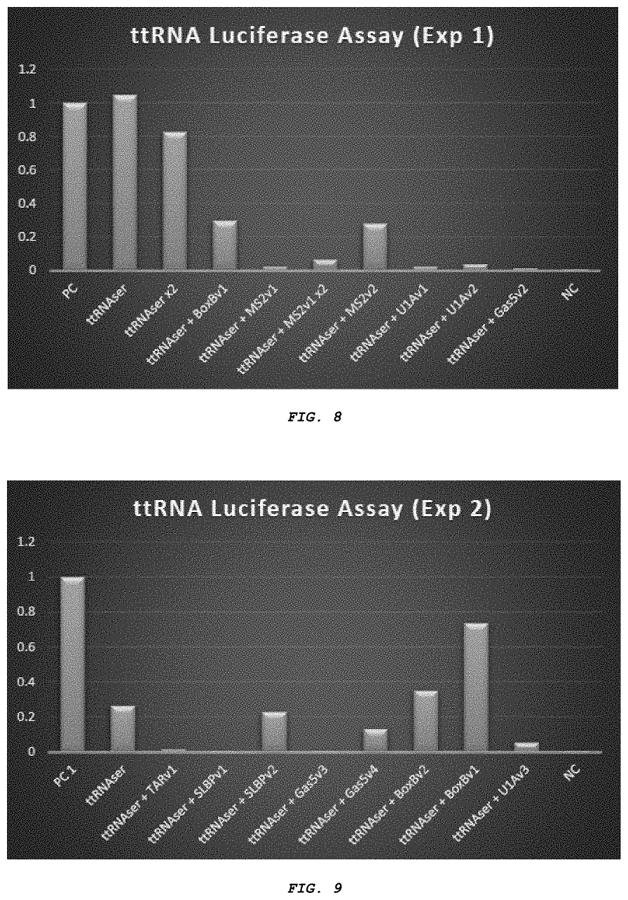

Additional experiments expanded on the above experiment in two ways (see, FIG. 7G). First, the amount of signal that the reporters were generating was quantified to better elucidate how structural changes to variable loop effect the ttRNA performance. In order to achieve this, a new luciferase (cLuc) reporter was cloned into a pCAG plasmid. Similarly, to the previous GFP reporter, a serine codon was replaced with a TAG PTC (s56x). The resulting reporter was compatible with the previous constructs and had luminescence that could be easily quantified. Second, the secondary structure of the ttRNA^{Ser-TAG}+BoxBv1 construct was analyzed in order to identify regions that maintain functionality. It was surmised that the particular G-C content within the stem of RNA hairpin loop was providing structural stability. The other constructs were modified in a similar manner and then cloned back into the HU6/MU6 plasmid. Once the next generation of constructs were prepared, they were transfected into HEK293 cells in the same manner as outlined in the previous experiment. FIG. 8 provides a plot of the results of this assay.

To clarify the naming conventions in FIG. 8, it should be noted that the ttRNA backbone is ttRNA^{Ser-TAG} which has the modified anti-codon loop for PTC suppression. The ttRNA constructs with the variable loop modification (i.e., ttRNAser+BoxBv1) also have the anti-codon loop modification for suppression.

All ttRNA constructs with variable loop modifications elicited improved signal recovery over background. The lowest performing was ttRNA^{Ser-TAG}+Gas5v2 with a ˜2-fold increase in signal over the negative control, this was likely due the relatively large RNA hairpin sequence, which can be observed in FIG. 6. While ttRNA^{Ser-TAG}+BoxBv1 had the highest signal recovery at about ˜30% versus the positive control. This reconfirms what was seen in the first experiment. Additionally, the structural hypothesis was confirmed with the signal observed by ttRNA^{Ser-TAG} MS2v2. A significant improvement between the two versions of this construct was noticed, which can be attributed to the additional sequence and structure modifications.

In order to test whether secondary structure of the ttRNA impacts signal recovery, some additional modifications to the existing constructs were made. The strategy to optimize the structure was to emulate the native variable loop of the tRNA^{Ser}. This usually resulted in shortening of the stem of the RNA hairpin, while maintaining the stability of the stem-loop. In addition to these modifications some new constructs were created which have affinity for human derived RNA binding proteins. A follow-on luciferase assay similar to FIG. 8 was performed, the results can be found in FIG. 9.

The foregoing studies demonstrates the potential efficacy of the suppressor ttRNA approach. While the visual signal shown in FIG. 7C may have been expected from the previous screens done on tRNA Ser(UAG) shown in FIG. 1, the most exciting and unexpected result was presented in FIG. 7D, which included the ttRNA^{Ser-TAG}+BoxBv1 construct. This is the first time a suppressor tRNA has been so heavily modified while still maintaining suppressor functionality. This is important, because it provides the opportunity to use this construct in the manner described in FIG. 5, and marks a notable improvement in the technological field. Of particular interest with these results is the increased signal recovery with each ttRNA construct version. With the exception of ttRNA^{Ser}+BoxBv2, each step of the construct version offers higher signal recovery. This suggests that the better the emulation of the native tRNA^{Ser} structure, the higher the efficiency of the construct. Additionally, several human derived RNA hairpins were identified with signal recovery making viable candidates to recruit endogenous RNA binding proteins. This marks an important step towards reduction to practice.

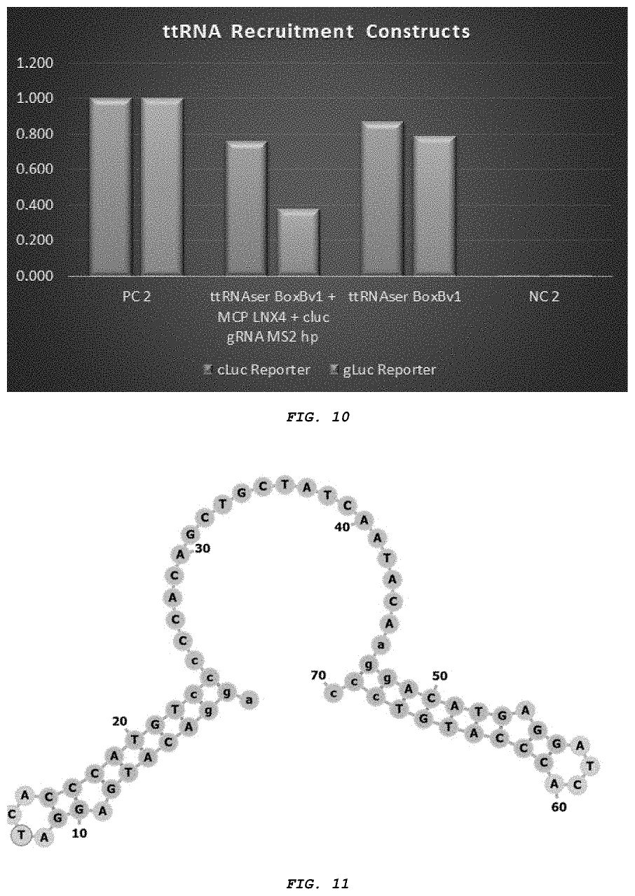

With the signal recovery data from a variety of constructs, the system described in FIG. 5 could be tested. The system comprises four components. The first component comprises the reporter plasmid, which is the same pCAG cLuc (S56X). The second component comprises a HU6/MU6 plasmid which contains the MS2 guide RNA (gRNA) and the protein complex MCP+LNx4. The MS2 gRNA will bind upstream of the TAG PTC found in the reporter, and serve as a binding site for MCP+LNx4 RNA binding proteins on the mRNA. The third component comprises a HU6/MU6 plasmid containing ttRNA^{Ser}+BoxBv1. This particular construct was chosen as showed significant signal recovery in previous experiments. The fourth component comprises a pCAG gLuc (S78X) reporter plasmid. This reporter plasmid provides information on whether efficiency was improved or whether the additional specificity negatively impacts the system versus previous findings. It was postulated that because the gRNA and protein complex are not designed for the gLuc reporter, relative signal differences should be measured between cLuc and gLuc. The results can be found below in FIG. 10.

It will be understood that various modifications may be made without departing from the spirit and scope of this disclosure. Accordingly, other embodiments are within the scope of the following claims.

What is claimed is:

1. A targeting transfer RNA (ttRNA) comprising:
a polynucleotide having a general structure from 5′ to 3′ of: 5′-(D-Loop domain)-(anticodon loop domain)-(variable loop domain)-(T loop domain)-3′,
wherein the anticodon loop domain comprises a sequence selected from N₁UCTAN₂N₃; N₁UUUAN₂N₃; or N₁UUCAN₂N₃, wherein N₁ is a pyrimidine, N₂ is a purine and N₃ is any of U, A or C, that binds to a stop codon sequence of an mRNA,
wherein the variable loop domain is at least about 12 to about 30 nucleotides in length and which comprises a sequence that is selectively bound by a protein or an agent tag, and
wherein the ttRNA at least partially suppresses nonsense mutations in messenger RNA.

2. The ttRNA of claim 1, wherein the variable loop is from 12 to 30 nucleotides in length.

3. The ttRNA of claim 1, wherein the ttRNA comprises a nucleic acid sequence that binds with an RNA binding protein.

4. The ttRNA of claim 1 or claim 3, wherein the polynucleotide is engineered from a tRNA for an amino acid selected from the group consisting of alanine, asparagine, aspartic acid, arginine, cysteine, glutamine, glycine, glutamic acid, histidine, isoleucine, lysine, leucine, phenylalanine, proline, methionine, serine, tryptophan, threonine, tyrosine, and valine.

5. The ttRNA of any one of the preceding claims, wherein the polynucleotide is engineered from a tRNA for serine or arginine.

6. The ttRNA of claim 4 or claim 5, wherein the tRNA is a tRNA gene found in a human genome.

7. The ttRNA of any one of the preceding claims, wherein the ttRNA has a cloverleaf-like structure.

8. The ttRNA of any one of the preceding claims, wherein the D-Loop domain and T-Loop domain of the polynucleotide comprises a naturally occurring sequence from a human tRNA.

9. The ttRNA of any one of the preceding claims, wherein the anticodon loop domain binds to a stop codon having a DNA sequence of ‘TAG’ or an RNA sequence of ‘UAG.’

10. The ttRNA of any one of claims 1 to 8, wherein the anticodon loop domain binds to a stop codon having a DNA sequence of ‘TAA’ or an RNA sequence of ‘UAA.’

11. The ttRNA of any one of claims 1 to 8, wherein the anticodon loop domain binds to a stop codon having a DNA sequence of ‘TGA’ or an RNA sequence of ‘UGA.’

12. The ttRNA of any one of the preceding claims, the variable loop domain comprises a sequence that is selectively bound by an RNA binding protein.

13. The ttRNA of claim 12, wherein the variable loop domain comprises a sequence for TBP, BoxBv1, BoxBv2, MS2v2, U1Av3, Gas5v4, TARv1, SLBPv1, or SLBPv2.

14. The ttRNA of any one of the preceding claims, where the variable loop domain comprises a sequence for BoxBv1.

15. The ttRNA of claim 12, the variable loop domain comprising a sequence with at least 70% sequence identity to a sequence selected from the group consisting of GGCCCTGAAAAAGGGCC, GGGACATGAGGATCACCCATGTCCC, AGCTTATCCATTGCACTCCGGATAAGCT, GGCCCAGTGGTCTTTGTAGACTGCCTGATGGCC, GGCCAGATCTGAGCCTGGGAGCTCTCTGGCC, CCAAAGGCTCTTCTCAGAGCCACCCA, and GGCTCTTCTCAGAGCC.

16. The ttRNA of any one of the preceding claims, where the polynucleotide comprises a sequence with at least 70% sequence identity to a sequence selected from (1) to (8):
(1) GTAGTCGTGGCCGAGTGGTTAAGGCGATGGACTCTAAATCCATT
GGCCCTGAAAAAGGGCCGCGCAGGTTCGAATCCTGCCGACTACG;
(2) GTAGTCGTGGCCGAGTGGTTAAGGCGATGGACTCTAAATCCATT
CGGCCCTGAAAAAGGGCCGCGCAGGTTCGAATCCTGCCGACTACG;
(3) GTAGTCGTGGCCGAGTGGTTAAGGCGATGGACTCTAAATCCATT
GGGGACATGAGGATCACCCATGTCCCGCGCAGGTTCGAATCCTGCCGA
CTACG;
(4) GTAGTCGTGGCCGAGTGGTTAAGGCGATGGACTCTAAATCCATT
AGCTTATCCATTGCACTCCGGATAAGCTGCGCAGGTTCGAATCCTGCC
GACTACG;
(5) GTAGTCGTGGCCGAGTGGTTAAGGCGATGGACTCTAAATCCATT
GGCCCAGTGGTCTTTGTAGACTGCCTGATGGCCGCGCAGGTTCGAATC
CTGCCGACTACG;
(6) GTAGTCGTGGCCGAGTGGTTAAGGCGATGGACTCTAAATCCATT
GGCCAGATCTGAGCCTGGGAGCTCTCTGGCCGCGCAGGTTCGAATCCT
GCCGACTACG;
(7) GTAGTCGTGGCCGAGTGGTTAAGGCGATGGACTCTAAATCCATT
CCAAAGGCTCTTCTCAGAGCCACCCAGCGCAGGTTCGAATCCTGCCGA
CTACG;
and
(8) GTAGTCGTGGCCGAGTGGTTAAGGCGATGGACTCTAAATCCATT
GGCTCTTCTCAGAGCCGCGCAGGTTCGAATCCTGCCGACTACG.

17. The ttRNA of any one of the preceding claims, wherein the polynucleotide comprises the sequence of:
GTAGTCGTGGCCGAGTGGTTAAGGCGATGGACTCTAAATCCATTGGCC
CTGAAAAAGGGCCGCGCAGGTTCGAATCCTGCCGACTACG.

18. A vector that encodes the ttRNA of any one of the preceding claims.

19. The vector of claim 18, wherein the vector is a viral vector.

20. The vector of claim 19, wherein the viral vector is a retroviral vector or an adeno-associated viral vector.

21. A composition for suppressing nonsense mutations in messenger RNA comprising:
the ttRNA of any one of claims 1 to 17, or the vector of any one of claims 18 to 20; and
a guide RNA (gRNA) that specifically binds to a nucleotide sequence about 20 to 100 bps from a nonsense mutation, wherein the gRNA further comprises one or more RNA binding protein sequences that have strong binding affinity for one or more RNA binding protein(s); and
one or more complexes comprising two RNA binding proteins that are fused together, wherein a first RNA binding protein of a complex has strong binding affinity for one or more RNA binding protein sequences of the gRNA, and wherein a second RNA binding protein of a complex has strong binding affinity for a sequence in the variable loop domain of the ttRNA.

22. The composition of claim 21, wherein the one or more complexes comprise two RNA binding proteins selected from the group consisting of LN, MCP, U1A, GRD-BD, TBP6.7, SLBP and variants thereof.

23. The composition of claim 21 or claim 22, wherein the one or more complexes comprise TBP and SLBPx3 fused together.

24. The composition of any one of claim 21 to claim 23, wherein the one or more complexes comprise MCP and LNx4 fused together.

25. A method for restoring translation in a cell of a nucleotide sequence that has a nonsense mutation comprising:
contacting the cell with a ttRNA of any one of claims 1 to 16, a vector of any one of claims 18 to 20, or a composition of any one of claims 21 to 24.

26. The method of claim 25, wherein the cell is contacted in vivo, in vitro, or ex vivo.

27. The method of claim 25 or claim 26, wherein the cell is from a subject that has a disorder associated with a nonsense mutation.

28. The method of claim 27, where the subject has a disorder selected from the group consisting of Duchenne muscular dystrophy, cystic fibrosis, Beta thalassaemia (β-globin), Hurler syndrome, Dravet Syndrome, and any combination thereof.

29. The method of claim 28, wherein the subject has Duchenne muscular dystrophy or cystic fibrosis.

30. An engineered targeting transfer RNA (ttRNA) comprising a modified variable loop, wherein the modified variable loop comprises a hairpin loop that binds to an RNA binding protein to a greater extent than a tRNA comprising an unmodified variable loop

31. The engineered ttRNA of claim 30, wherein the tRNA comprising the unmodified variable loop is naturally present in a human cell.

32. The engineered ttRNA of claim 30, wherein the hairpin loop comprises at least a portion of an aptamer.

33. The engineered ttRNA of claim 30, wherein the hairpin loop comprises at least a portion of an MS2 domain.

34. The engineered ttRNA of claim 30, wherein the hairpin loop comprises at least a portion of a BoxB domain.

35. The engineered ttRNA of claim 30, wherein the hairpin loop comprises at least a portion of a U1hpII domain.

36. The engineered ttRNA of claim 30, wherein the hairpin loop comprises at least a portion of a Gas5 domain.

37. The engineered ttRNA of claim 30 or 31, wherein the RNA binding protein is selected from the group consisting of at least a portion of: Lambda N, MCP, U1A, GR-DBD, and any combination thereof.

38. The engineered ttRNA of any one of claims 30-32, wherein the engineered ttRNA is acylated with an amino acid.

39. The engineered ttRNA of claim 38, wherein the amino acid is a canonical amino acid or a non-canonical amino acid.

40. The engineered ttRNA of claim 39, wherein the amino acid is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

41. The engineered ttRNA of any one of claims 30-40, wherein the engineered ttRNA comprises an anticodon loop that recognizes a stop codon implicated in a disease or condition.

42. The engineered ttRNA of claim 41, wherein the stop codon is a premature stop codon.

43. The engineered ttRNA of claim 41, wherein the stop codon is UAA.

44. The engineered ttRNA of claim 41, wherein the stop codon is UGA.

45. The engineered ttRNA of claim 41, wherein the stop codon is UAG.

46. The engineered ttRNA of any one of claims 30-43, wherein the engineered ttRNA when contacted with the target RNA at least partially disrupts association of a release factor protein with the target RNA.

47. The engineered ttRNA of any one of claims 30-46, wherein the engineered ttRNA comprises an anticodon loop that recognizes a sense codon, wherein the sense codon comprises the mutation in the target RNA that is implicated in the disease or condition.

48. The engineered ttRNA of claim 47, wherein the sense codon is GCU, GCC, GCA, GCG, CGU, CGC, CGA, CGG, AGA, AGG, AAU, AAC, GAU, GAC, UGU, UGC, GAA, GAG, CAA, CAG, GGU, GGC, GGA, GGG, CAU, CAC, AUU, AUC, AUA, UUA, UUG, CUU, CUC, CUA, CUG, AAA, AAG, AUG, UUU, UUC, CCU, CCC, CCA, CCG, UCU, UCC, UCA, UCG, AGU, AGC, ACU, ACC, ACA, ACG, UGG, UAU, UAC, GUU, GUC, GUA, or GUG.

49. The engineered ttRNA of any one of claims 43-45, wherein the engineered ttRNA when contacted with the target RNA at least partially disrupts association of a tRNA naturally present in a human with the target RNA, wherein the tRNA naturally present in the human cell comprises an anticodon loop that is complementary to the sense codon.

50. The engineered ttRNA of any one of claims 40-45, wherein at least one nucleotide of the engineered ttRNA is a chemically modified nucleotide.

51. The engineered ttRNA of any one of claims 40-45, wherein the engineered ttRNA comprises a sugar modification.

52. The engineered ttRNA of any one of claims 30-49, wherein a nucleotide of the engineered ttRNA comprises a methyl group, a fluoro group, a methoxyethyl group, an ethyl group, a phosphate group, an amide group, an ester group, or any combination thereof.

53. The engineered ttRNA of any one of claims 30-50, wherein the engineered ttRNA is genetically encodable.

54. The engineered ttRNA of any one of claims 30-51, wherein the engineered ttRNA comprises a reducing 3′ hydroxyl group.

55. The engineered ttRNA of any one of claims 30-54, wherein the modified variable loop comprises a hairpin loop that binds to an RNA binding protein to a greater extent than a tRNA comprising an unmodified variable loop is determined by an in vitro assay.

56. The engineered ttRNA of any one of claims 30-55, wherein the disease or condition comprises Rett Syndrome, Duchenne Muscular Dystrophy, Stargardt's Syndrome, or any combination thereof.

57. A system comprising:
(a) the engineered ttRNA of any one of claims 30-56,
(b) a first RNA binding protein, and
(c) a guide RNA comprising an antisense domain, wherein the antisense domain is complementary to at least a portion of a target RNA comprising a mutation, and wherein the mutation of the target RNA is implicated in a disease or condition.

58. The system of claim 57, wherein the engineered ttRNA further comprises a hairpin loop that binds to a second RNA binding protein.

59. The system of claim 58, wherein the first RNA binding protein, the second RNA binding protein, or both is independently selected from the group consisting of at least a portion of: Lambda N, MCP, U1A, GR-DBD, and any combination thereof.

60. The system of any one of claims 57-59, wherein the guide RNA comprises a hairpin capable of binding the first RNA binding protein.

61. The system of any of one of claim 58, wherein the hairpin comprises a BoxB hairpin.

62. The system of any one of claims 55-61, wherein the engineered ttRNA is capable of binding the first RNA binding protein.

63. The system of any one of claim 60, wherein the hairpin comprises a MS2 hairpin.

64. The system of any one of claims 57-62, wherein the first RNA binding protein comprises a first binding domain that binds a second binding domain of the second RNA binding protein.

65. The system of claim 64, wherein the first RNA binding protein binds the second RNA binding protein with a K_{D} of from about 1 nM to about 100 μM.

66. The system of any one of claim 57-65, wherein the guide RNA forms a secondary structure comprising: a stem loop, a cruciform, a toe hold, a mismatch, or any combination thereof.

67. The system of any one of claims 57-66, wherein the RNA binding protein is delivered exogenously.

68. The system of any one of claims 57-67, wherein the RNA binding protein is endogenously expressed.

69. A vector comprising a polynucleotide sequence encoding for the engineered ttRNA of any one of claims 30-56 or the engineered ttRNA and guide RNA of the system of any one of claims 57-68.

70. The vector of claim 69, wherein the vector comprises a liposome, a viral vector, a nanoparticle, or any combination thereof.

71. The vector of claim 69, wherein the vector is the viral vector, and wherein the viral vector is an AAV vector.

72. An isolated cell that comprises the engineered ttRNA of any one of claims 30-56, the system of any one of claims 57-68, or the vector of any one of claims 69-71.

73. A pharmaceutical composition in unit dose form comprising:
(a) the engineered ttRNA of any one of claims 30-56, the engineered ttRNA and guide RNA of the system of any one of claims 57-68, or the vector of any one of claims 69-71, and
(b) a pharmaceutically acceptable: excipient, diluent, or carrier.

74. A method of at least partially ameliorating or preventing a disease or condition in a subject in need thereof comprising: administering to the subject the engineered ttRNA of any one of claims 30-56, the engineered ttRNA and guide RNA of the system of any one of claims 57-68, the vector of any one of claims 69-71, or the pharmaceutical composition of claim 70, and wherein the administering is sufficient at least partially ameliorate the disease or condition in the subject.

75. The method of claim 74, wherein the administering is by intravenous injection, intramuscular injection, an intrathecal injection, an intraorbital injection, a subcutaneous injection, or any combination thereof.

76. The method of claim 74 or 75, wherein the disease or condition is selected from the group consisting of: a neurodegenerative disorder, a muscular disorder, a metabolic disorder, an ocular disorder, a cancer, and any combination thereof.

77. The method of any one of claims 74-76, wherein the disease or condition comprises Rett Syndrome.

78. The method of any one of claims 74-76, wherein the subject is a mammal.

79. The method of claim 78, wherein the mammal is a human.

80. The method of claim 79, wherein the mammal is a non-human mammal.

81. The method of any one of claims 74-80, wherein the subject has been diagnosed with the disease or condition by an in vitro diagnostic.

82. A kit comprising: the engineered ttRNA of any one of claims 30-56 in a container, the engineered ttRNA and guide RNA of the system of any one of claims 64-68 in a container, or the vector of any one of claims 69-71 in a container.

83. A method of making a kit, comprising placing at least in part, into a container:
the engineered ttRNA of any one of claims 30-56, the engineered ttRNA and guide RNA of the system of any one of claims 57-68, or the vector of any one of claims 69-71.

84. A method of making a pharmaceutical composition, comprising contacting a pharmaceutically acceptable: excipient, carrier, or diluent with at least one of the engineered ttRNA of any one of claims 30-56, the engineered ttRNA and guide RNA of the system of any one of claims 57-68, or the vector of any one of claims 69-71.


SEQUENCE LISTING (33 sequences)

SEQ ID NO: 1 - RNA, 21 nt, Artificial Sequence
  Random template illustration
uucgauugguaguucgauugg

SEQ ID NO: 2 - protein, 7 aa, Artificial Sequence
  Random template illustration
FDWSFDW

SEQ ID NO: 3 - DNA, 82 bp, Artificial Sequence
  Ser tRNA
gtagtcgtggccgagtggttaaggcgatggactagaaatccattggggtttccccgcgcaggttcgaatcctgccgactacg

SEQ ID NO: 4 - DNA, 82 bp, Artificial Sequence
  Modified tRNA Ser-TAG
gtagtcgtggccgagtggttaaggcgatggactctaaatccattggggtttccccgcgcaggttcgaatcctgccgactacg

SEQ ID NO: 5 - DNA, 88 bp, Artificial Sequence
  ttRNA Ser-TAG-BoxBv1
gtagtcgtggccgagtggttaaggcgatggactctaaatccattggccctgaaaaagggccgcgcaggttcgaatcctgccgactacg

SEQ ID NO: 6 - DNA, 89 bp, Artificial Sequence
  ttRNA Ser-TAG-BoxBv2
gtagtcgtggccgagtggttaaggcgatggactctaaatccattgggccctgaaaaagggccgcgcaggttcgaatcctgccgactacg

SEQ ID NO: 7 - DNA, 97 bp, Artificial Sequence
  ttRNA Ser-TAG MS2v2
gtagtcgtggccgagtggttaaggcgatggactctaaatccattggggacatgaggatcacccatgtcccgcgcaggttcgaatcctgccgactacg

SEQ ID NO: 8 - DNA, 100 bp, Artificial Sequence
  ttRNA Ser-TAG-U1Av3
gtagtcgtggccgagtggttaaggcgatggactctaaatccattgagcttatccattgcactccggataagctgcgcaggttcgaatcctgccgactacg

SEQ ID NO: 9 - DNA, 105 bp, Artificial Sequence
  ttRNA Ser-TAG-Gas5v4
gtagtcgtggccgagtggttaaggcgatggactctaaatccattgggcccagtggtctttgtagactgcctgatggccgcgcaggttcgaatcctgccgactacg

SEQ ID NO: 10 - DNA, 103 bp, Artificial Sequence
  ttRNA Ser-TAG-TARv1
gtagtcgtggccgagtggttaaggcgatggactctaaatccattgggccagatctgagcctgggagctctctggccgcgcaggttcgaatcctgccgactacg

SEQ ID NO: 11 - DNA, 98 bp, Artificial Sequence
  ttRNA Ser-TAG-SLBPv1
gtagtcgtggccgagtggttaaggcgatggactctaaatccattgccaaaggctcttctcagagccacccagcgcaggttcgaatcctgccgactacg

SEQ ID NO: 12 - DNA, 88 bp, Artificial Sequence
  ttRNA Ser-TAG-SLBPv2
gtagtcgtggccgagtggttaaggcgatggactctaaatccattgggctcttctcagagccgcgcaggttcgaatcctgccgactacg

SEQ ID NO: 13 - DNA, 70 bp, Artificial Sequence
  cLuc gRNA with MS2 Hairpin
aggacatgaggatcacccatgtcccccacagctgctatcaatacaaggacatgaggatcacccatgtccc

SEQ ID NO: 14 - RNA, 15 nt, Artificial Sequence
  Box B sequence
gcccugaaaaagggc

SEQ ID NO: 15 - RNA, 17 nt, Artificial Sequence
  BoxB sequence
ggcccugaaaaagggcc

SEQ ID NO: 16 - DNA, 20 bp, Artificial Sequence
  Csy4 target coding sequence
gttcactgccgtataggcag

SEQ ID NO: 17 - RNA, 28 nt, Artificial Sequence
  Csy4 RNA target sequence
guucacugccguauaggcagcuaagaaa

SEQ ID NO: 18 - RNA, 6 nt, Artificial Sequence
  ttRNA anticodon consensus sequence
  misc_feature (1)..(1): N is a pyrimidine
  misc_feature (5)..(5): n is a, c, g, or u
  misc_feature (6)..(6): N is a purine
  misc_feature (7)..(7): N is U, A or C
nucann

SEQ ID NO: 19 - RNA, 7 nt, Artificial Sequence
  ttRNA anticodon consensus sequence
  misc_feature (1)..(1): N is a pyrimidine
  misc_feature (6)..(6): N is a purine
  misc_feature (7)..(7): N is U, A or C
nuuuann

SEQ ID NO: 20 - RNA, 7 nt, Artificial Sequence
  ttRNA anticodon consensus sequence
  misc_feature (1)..(1): N is a pyrimidine
  misc_feature (6)..(6): N is a purine
  misc_feature (7)..(7): N is U, A or C
nuucann

SEQ ID NO: 21 - DNA, 17 bp, Artificial Sequence
  Oligonucleotide coding motif for LambdaN
ggccctgaaaaagggcc

SEQ ID NO: 22 - DNA, 25 bp, Artificial Sequence
  Oligonucleotide coding motif for MCP
gggacatgaggatcacccatgtccc

SEQ ID NO: 23 - DNA, 28 bp, Artificial Sequence
  Oligonucleotide coding motif for U1A
agcttatccattgcactccggataagct

SEQ ID NO: 24 - DNA, 33 bp, Artificial Sequence
  Oligonucleotide coding motif for GR-DBD
ggcccagtggtctttgtagactgcctgatggcc

SEQ ID NO: 25 - DNA, 31 bp, Artificial Sequence
  Oligonucleotide coding motif for TBP6.7
ggccagatctgagcctgggagctctctggcc

SEQ ID NO: 26 - DNA, 26 bp, Artificial Sequence
  Oligonucleotide coding motif for SLBP
ccaaaggctcttctcagagccaccca

SEQ ID NO: 27 - DNA, 16 bp, Artificial Sequence
  Oligonucleotide coding motif for SLBP
ggctcttctcagagcc

SEQ ID NO: 28 - RNA, 95 nt, Artificial Sequence
  Primate tRNA consensus sequence isotype Ala
  misc_feature (17)..(23): n is either present or absent and if present n is any RNA base
  misc_feature (31)..(31): n is a paired consensus sequence A/T, A/U or G/C
  misc_feature (33)..(33): n is a paired consensus sequence A/T, A/U or G/C
  misc_feature (43)..(43): n is a paired consensus sequence A/T, A/U or G/C
  misc_feature (45)..(45): n is a paired consensus sequence A/T, A/U or G/C
  misc_feature (49)..(67): n is either present or absent and if present n is any RNA base
ggggrwkuagcucardnnggunnagagcgcnyncuuhgcaugnrngagnnnnnnnnnnnnnnnnnnngymsygggwucrayscccrsmwycucca

SEQ ID NO: 29 - RNA, 95 nt, Artificial Sequence
  Primate tRNA consensus sequence isotype Arg
  misc_feature (1)..(2): n is a paired consensus sequence A/T, A/U or G/C
  misc_feature (5)..(6): n is a paired consensus sequence A/T, A/U or G/C
  misc_feature (13)..(13): n is either present or absent and if present n is any RNA base
  misc_feature (17)..(17): n is either present or absent and if present n is any RNA base
  misc_feature (18)..(18): n is either present or absent and if present n is any RNA base
  misc_feature (23)..(23): n is either present or absent and if present n is any RNA base
  misc_feature (25)..(25): n is either present or absent and if present n is any RNA base
  misc_feature (30)..(30): n is a paired consensus sequence A/T, A/U or G/C
  misc_feature (33)..(33): n is a paired consensus sequence A/T, A/U or G/C
  misc_feature (43)..(43): n is a paired consensus sequence A/T, A/U or G/C
  misc_feature (46)..(46): n is a paired consensus sequence A/T, A/U or G/C
  misc_feature (49)..(67): n is either present or absent and if present n is any RNA base
  misc_feature (71)..(72): n is a paired consensus sequence A/T, A/U or G/C
  misc_feature (86)..(87): n is a paired consensus sequence A/T, A/U or G/C
  misc_feature (89)..(90): n is a paired consensus sequence A/T, A/U or G/C
  misc_feature (93)..(94): n is a paired consensus sequence A/T, A/U or G/C
nnsynnguggcsnaaunnggaunansgcrnyknmcuhckraknmrnrgnnnnnnnnnnnnnnnnnnnruunnrgguucgavuccynncnnrsnnr

SEQ ID NO: 30 - RNA, 95 nt, Artificial Sequence
  Primate tRNA consensus sequence isotype Asn
  misc_feature (1)..(1): n is a paired consensus sequence A/T, A/U or G/C
  misc_feature (4)..(4): n is a paired consensus sequence A/T, A/U or G/C
  misc_feature (12)..(12): n is a paired consensus sequence A/T, A/U or G/C
  misc_feature (23)..(23): n is either present or absent and if present n is any RNA base
  misc_feature (26)..(26): n is a paired consensus sequence A/T, A/U or G/C
  misc_feature (31)..(33): n is a paired consensus sequence A/T, A/U or G/C
  misc_feature (43)..(45): n is a paired consensus sequence A/T, A/U or G/C
  misc_feature (49)..(67): n is either present or absent and if present n is any RNA base
  misc_feature (91)..(91): n is a paired consensus sequence A/T, A/U or G/C
  misc_feature (94)..(94): n is a paired consensus sequence A/T, A/U or G/C
nucncuguggcncaauysggyunagngcgunnngcuguuaacnnnaagnnnnnnnnnnnnnnnnnnnguuggugguucradcccacccagngang

SEQ ID NO: 31 - RNA, 95 nt, Artificial Sequence
  Primate tRNA consensus sequence isotype Asp
  misc_feature (4)..(4): n is a paired consensus sequence A/T, A/U or G/C
  misc_feature (6)..(6): n is a paired consensus sequence A/T, A/U or G/C
  misc_feature (17)..(17): n is either present or absent and if present n is any RNA base
  misc_feature (18)..(18): n is either present or absent and if present n is any RNA base
  misc_feature (23)..(23): n is either present or absent and if present n is any RNA base
  misc_feature (33)..(33): n is a paired consensus sequence A/T, A/U or G/C
  misc_feature (43)..(43): n is a paired consensus sequence A/T, A/U or G/C
  misc_feature (49)..(69): n is either present or absent and if present n is any RNA base
  misc_feature (89)..(89): n is a paired consensus sequence A/T, A/U or G/C
  misc_feature (91)..(91): n is a paired consensus sequence A/T, A/U or G/C
uccncnuuaguauagunngguknaguauycccnccugucacgngggagnnnnnnnnnnnnnnnnnnnanccsggguucrauucccsgangnggag

SEQ ID NO: 32 - RNA, 95 nt, Artificial Sequence
  Primate tRNA consensus sequence isotype Cys
  misc_feature (7)..(7): n is a paired consensus sequence A/T, A/U or G/C
  misc_feature (17)..(18): n is either present or absent and if present n is any RNA base
  misc_feature (22)..(23): n is either present or absent and if present n is any RNA base
  misc_feature (49)..(67): n is either present or absent and if present n is any RNA base
  misc_feature (73)..(73): n is a paired consensus sequence A/T, A/U or G/C
  misc_feature (85)..(85): n is a paired consensus sequence A/T, A/U or G/C
  misc_feature (88)..(88): n is a paired consensus sequence A/T, A/U or G/C
gggggunuagcucagbnnggunnagagcauuugacugcagaucaagagnnnnnnnnnnnnnnnnnnngucccnrguucaaaucynggngcccccu

SEQ ID NO: 33 - RNA, 95 nt, Artificial Sequence
  Primate tRNA consensus sequence isotype Gln
  misc_feature (3)..(3): n is either present or absent and if present n is any RNA base
  misc_feature (17)..(18): n is either present or absent and if present n is any RNA base
  misc_feature (23)..(23): n is either present or absent and if present n is any RNA base
  misc_feature (49)..(67): n is either present or absent and if present n is any RNA base
  misc_feature (69)..(69): n is either present or absent and if present n is any RNA base
  misc_feature (92)..(92): n is either present or absent and if present n is any RNA base
ggnyccaugguguaaunngguknagcacucuggacuyugaauccagyrnnnnnnnnnnnnnnnnnnnanuccgaguucrarucucgguggrnccu